(19) United States
(12) Patent Application Publication
(10) Pub. No.: US 2025/0161610 A1
(43) Pub. Date: May 22, 2025

(54) GASTRIC AND TRACHEAL TUBE HOLDING ASSEMBLIES

(71) Applicant: ASPISAFE SOLUTIONS INC., New York, NY (US)

(72) Inventor: Talal Sharaiha, New York, NY (US)

(73) Assignee: ASPISAFE SOLUTIONS INC., New York, NY (US)

(21) Appl. No.: 18/952,616

(22) Filed: Nov. 19, 2024

Related U.S. Application Data
(60) Provisional application No. 63/600,947, filed on Nov. 20, 2023.

(51) Int. Cl. A61M 16/04; A61J 15/00

(52) U.S. Cl. CPC A61M 16/0497; A61J 15/0061

(57) ABSTRACT

Tube holding assemblies and methods for using and making the same are provided, including an assembly for holding a first tube (e.g., a gastric (e.g., nasogastric or orogastric) or tracheal (e.g., endotracheal (e.g., nasotracheal or orotracheal)) tube) to a patient's head while also being adjustable along a track that may be positioned underneath the patient's nose but above the patient's mouth and about an axis of rotation, as well as for simultaneously holding a second tube (e.g., a gastric (e.g., nasogastric or orogastric) or tracheal (e.g., endotracheal (e.g., nasotracheal or orotracheal)) tube) to the patient's head while also being adjustable along the same track and about another axis of rotation.

### CROSS-REFERENCE TO APPLICATION(S)

This application claims the benefit of prior filed U.S. Provisional Patent Application No. 63/600,947, filed Nov. 20, 2023, which is hereby incorporated by reference herein in its entirety.

### COPYRIGHT NOTICE

At least a portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyright rights whatsoever.

### Trademark Notice

Applicant(s) make no claim to any trademarks referenced in the disclosure.

### TECHNICAL FIELD

This disclosure relates to tube holding assemblies and, more particularly, to gastric and/or tracheal tube holding assemblies and methods for using and making the same.

### BACKGROUND OF THE DISCLOSURE

Various medical procedures (e.g., feeding and/or medicating and/or intubating procedures) involve a distal end of a tube being inserted into a specific area of a patient and then using the tube for injecting material into the patient and/or for removing material from the patient. However, safely securing such a tube at a particular position with respect to the patient during use has heretofore been difficult.

### SUMMARY OF THE DISCLOSURE

This document describes tube holding assemblies and methods for using and making the same.

For example, a method of using an assembly is provided for holding a tube and another tube to a patient's head once a distal end of the tube has been functionally positioned within the patient via a first nostril of a nose of the patient. The method may include positioning a mount subassembly of the assembly such that a track of the positioned mount subassembly extends above and along an upper lip of the patient, sliding a slider clamp body of a lock subassembly of the assembly along the track of the positioned mount subassembly until a tube clamp body of the lock subassembly is aligned with a portion of the tube, rotating the tube clamp body with respect to the slid slider clamp body until a passageway of the tube clamp body is aligned with the portion of the tube, placing the portion of the tube within the passageway of the rotated tube clamp body, and sliding another slider clamp body of another lock subassembly of the assembly along the track of the positioned mount subassembly until another tube clamp body of the other lock subassembly is aligned with at least one of a portion of the other tube, a second nostril of the nose of the patient, or a mouth of the patient. In some embodiments, the tube is a tracheal tube and the other tube is a gastric tube. In some embodiments, once a distal end of the other tube has been functionally positioned within the patient via the mouth of the patient, the sliding the other slider clamp body may include sliding the other slider clamp body of the other lock subassembly of the assembly along the track of the positioned mount subassembly until the other tube clamp body of the other lock subassembly is aligned with a portion of the other tube.

As another example, an assembly is provided for holding a tube and another tube to a patient's head. The assembly may include a mount subassembly defining a track, a lock subassembly including a slider clamp body operative to be coupled to and slidable along the track, a tube clamp body operative to receive and hold a portion of the tube in place with respect to the tube clamp body, and a hinge operative to allow rotation of the slider clamp body with respect to the tube clamp body about an axis of rotation, another lock subassembly including another slider clamp body operative to be coupled to and slidable along the track, another tube clamp body operative to receive and hold a portion of the other tube in place with respect to the other tube clamp body, and another hinge operative to allow rotation of the other slider clamp body with respect to the other tube clamp body about another axis of rotation, and a fastener subassembly operative to secure the mount subassembly to the patient's head. In some embodiments, when the slider clamp body is coupled to the track and the other slider clamp body is coupled to the track, the axis of rotation is above the track and the other axis of rotation is below the track. In some embodiments, when the slider clamp body is coupled to the track, the axis of rotation is above the track. In some embodiments, when the other slider clamp body is coupled to the track, the other axis of rotation is below the track. In some embodiments, the hinge is operative to allow at least 180 degree rotation of the slider clamp body with respect to the tube clamp body about the axis of rotation. In some embodiments, the other hinge is operative to allow at least 180 degree rotation of the other slider clamp body with respect to the other tube clamp body about the other axis of rotation. In some embodiments, the tube is a tracheal tube and the other tube is a gastric tube.

As another example, an assembly is provided for holding a tube and another tube to a patient's head. The assembly may include a mount subassembly including a base and a track extending from and along a front face of the base, a lock subassembly including a slider clamp body operative to be coupled to and slidable along the track, a tube clamp body operative to be coupled to the tube, and a hinge operative to allow movement of the slider clamp body with respect to the tube clamp body, another lock subassembly including another slider clamp body operative to be coupled to and slidable along the track, another tube clamp body operative to be coupled to the other tube, and another hinge operative to allow movement of the other slider clamp body with respect to the other tube clamp body, and a strap subassembly operative to secure the mount subassembly to the patient's head. In some embodiments, the tube clamp body defines a tube clamp passageway along and within which the tube clamp body is operative to hold a portion of the tube in place with respect to the tube clamp body, and, when the slider clamp body is coupled to the track, the assembly is operative to allow the tube clamp passageway to extend simultaneously transverse to the track and in front of the front face of the base. In some embodiments, the tube clamp body defines a tube clamp passageway along a length of an interior of the tube clamp body extending between a tube clamp body top and a tube clamp body bottom, the tube clamp body is operative to hold a portion of the tube in place with respect to the tube clamp body within the tube clamp passageway and along the length of the interior of the tube clamp body, the tube clamp passageway is accessible by the portion of the tube via a tube clamp body opening extending through a front face of the tube clamp body and between the tube clamp body top and the tube clamp body bottom, the tube clamp body opening is operative to introduce the portion of the tube into the tube clamp passageway, and, when the slider clamp body is coupled to the track, the hinge is operative to allow movement of the slider clamp body with respect to the tube clamp body such that the tube clamp passageway is positioned between the tube clamp body opening and the track. In some embodiments, the hinge is operative to allow movement of the slider clamp body with respect to the tube clamp body such that the tube clamp body opening faces away from the slider clamp body when the slider clamp body is coupled to the track. In some embodiments, the hinge is operative to allow movement of the slider clamp body with respect to the tube clamp body such that the tube clamp body opening faces away from the slider clamp body when the slider clamp body is coupled to the track. In some embodiments, the hinge is operative to allow rotation of the slider clamp body with respect to the tube clamp body about an axis of rotation. In some embodiments, the other hinge is operative to allow rotation of the other slider clamp body with respect to the other tube clamp body about another axis of rotation. In some embodiments, when the slider clamp body is coupled to the track and the other slider clamp body is coupled to the track, the axis of rotation is above the track and the other axis of rotation is below the track. In some embodiments, the hinge is operative to allow at least 180 degree rotation of the slider clamp body with respect to the tube clamp body about the axis of rotation. In some embodiments, the tube is a tracheal tube and the other tube is a gastric tube.

For example, an assembly for holding a tube (e.g., gastric (e.g., nasogastric or orogastric) or tracheal (e.g., endotracheal (e.g., nasotracheal or orotracheal)) tube) to a patient's head is provided that may include a mount subassembly including a base and a track extending from and along a front face of the base, a lock subassembly including a slider clamp body operative to be coupled to and slidable along the track, a tube clamp body operative to receive and hold a portion of the tube in place with respect to the tube clamp body, and a hinge operative to allow limited movement of the slider clamp body with respect to the tube clamp body, and a strap subassembly operative to secure the mount subassembly to the patient's head.

As another example, an assembly for holding a tube (e.g., gastric (e.g., nasogastric or orogastric) or tracheal (e.g., endotracheal (e.g., nasotracheal or orotracheal)) tube) to a patient's head is provided that may include a mount subassembly defining a track, a lock subassembly including a slider clamp body operative to be coupled to and slidable along the track, a tube clamp body operative to receive and hold a portion of the tube in place with respect to the tube clamp body, and a hinge operative to allow limited rotation of the slider clamp body with respect to the tube clamp body, and a fastener subassembly operative to secure the mount subassembly to the patient's head.

As yet another example, a method of using an assembly for holding a tube (e.g., gastric (e.g., nasogastric or orogastric) or tracheal (e.g., endotracheal (e.g., nasotracheal or orotracheal)) tube) to a patient's head once a distal end of the tube has been functionally positioned within the patient (e.g., via a nostril of a nose or via a mouth of the patient) is provided that may include positioning a mount subassembly of the assembly underneath the nose or mouth, sliding a slider clamp body of a lock subassembly of the assembly along a track of the positioned mount subassembly until a tube clamp body of the lock subassembly is aligned with a portion of the tube, rotating the tube clamp body with respect to the slid slider clamp body until a passageway of the tube clamp body is aligned with the portion of the tube, and placing the portion of the tube within the passageway of the rotated tube clamp body.

In yet another example, a device for holding a tube (e.g., gastric (e.g., nasogastric or orogastric) or tracheal (e.g., endotracheal (e.g., nasotracheal or orotracheal)) tube) tube to a patient has a track configured to fit adjacent a lip on a patient's face. The track has a face contacting side, an exposed side opposite the face contacting side, and a pair of opposite ends. A tube holder is coupled to and slidable along the track between the opposite ends. A positioning mechanism is releasably lockable to allow selective lateral repositioning of the tube holder and a tube held thereby along the track and to retain the tube holder at a selected position along the track. A bite block is carried by the tube holder and is slidable therewith along the track. The bite block has a tubular wall with a generally cylindrical shape, a central opening along a length of the bite block, and a pair of opposite open ends. The bite block is positioned and spaced vertically from the track and has a working end portion that extends in the direction of and further beyond the face contacting side of the track.

In one example, the bite block can have an insertion slot through the wall and along the entire length of the bite block. The wall can be sufficiently flexible to allow an endotracheal tube to be inserted laterally into the central opening through the insertion slot.

In one example, the bite block can have an insertion slot through the wall and along the entire length of the bite block. The insertion slot can be defined between spaced apart free edges of the wall that confront one another along the length of the bite block.

In one example, one of the free edges can have a first contoured shape and the other of the free edges can have a corresponding second contoured shape configured to close against and engage the first contoured shape if a sufficient clamping force is exerted on an outer surface of the bite block, such as if a critical care patient were to bite down on the bite block during use.

In one example, one of the free edges can have a convex rounded edge and the other of the free edges can have a concave rounded edge sized to receive the convex rounded edge.

In one example, one of the free edges can have an angled or beveled edge and the other of the free edges can have a corresponding angled or beveled edge arranged to engage and mate with one another.

In one example, one of the free edges can have a convex V-shaped protrusion and the other of the free edges can have a corresponding concave V-shaped groove configured to receive the convex V-shaped protrusion.

In one example, each of the free edges can have a flange extending inward into the central opening of the bite block and can be configured to close against and engage one another if a sufficient clamping force is exerted on an outer surface of the bite block.

In one example, each of the free edges can have a flange extending inward into the central opening of the bite block and can be configured to close against and engage one another. An accessory line channel can be formed or defined along an inner surface of the wall adjacent one or both of the flanges within the central opening of the bite block.

In one example, the bite block can have an insertion slot through the wall and along the entire length of the bite block. A living hinge or relief can be provided in the wall generally opposite the insertion slot and extending a length of the bite block. The living hinge can render the wall sufficiently flexible to allow an endotracheal tube to be inserted laterally into the central opening through the insertion slot.

In one example, a blind slot can be formed in the working end portion of the bite block and can extend only part way along the length of the wall of the working end portion.

In one example, the bite block can have an insertion slot through the wall and along the entire length of the bite block. A blind slot can be formed in the working end portion of the bite block and can extend only part way along the length of the wall of the working end portion and can be positioned generally opposite the insertion slot.

In one example, the device can have an accessory line channel extending the length of the bite block and formed between an inner surface of the wall and an outer surface of an endotracheal tube positioned within the central opening of the bite block.

In one example, an accessory line channel can be formed between two spaced apart ribs extending the length of the bite block on an inner surface of the wall.

In one example, an accessory line channel can be formed as a V-shaped groove extending along and recessed into an inner surface of the wall.

In one example, the track can have a lip pad on the face contacting side.

In one example, the track can be configured to rest above an upper lip of a patient and the bite block can be spaced below the track.

In one example, the device can include a cheek plate connected to each of the opposite ends of the track, a cheek pad coupled to each of the cheek plates, an adhesive layer on a face contacting side of each pad, and an adjustable head strap coupled to the device for securing the device to a patient's head and retaining the track, cheek plates, and cheek pads on the patient's face.

In one example, the tube holder can have a tube strap configured to wrap around and secure an endotracheal tube to the tube holder.

In one example, the tube holder can have a tube strap configured to wrap around and secure an endotracheal tube to the tube holder. A surface of the tube strap can include an adhesive that contacts an outer surface of an endotracheal tube when the tube strap is wrapped around the endotracheal tube.

In one example, the device can include retention spikes protruding from a surface of the tube holder and positioned to contact and impinge on an outer surface of an endotracheal tube when attached to the tube holder.

In one example according to the teachings of the present disclosure, a tube securing device (e.g., an endotracheal tube securing device) has a track configured to fit above an upper lip on a patient's face. The track has a face contacting side, an exposed side opposite the face contacting side, and a pair of opposite ends. A tube holder is coupled to and slidable along the track between the opposite ends. A positioning mechanism is releasably lockable to allow selective lateral repositioning of the tube holder and a tube (e.g., gastric (e.g., nasogastric or orogastric) or tracheal (e.g., endotracheal (e.g., nasotracheal or orotracheal)) tube) held thereby along the track and to retain the tube holder at a selected position along the track. An adjustable head strap or multiple straps are coupled to the device for securing the device to a patient's head and retaining the track on the patient's face. A cheek plate is connected to each of the opposite ends of the track. Each cheek plate carries a cheek pad with an adhesive layer on a face contacting side of the cheek pad. A bite block is integrally molded as part of the tube holder and is slidable therewith along the track. The bite block has a tubular wall with a generally cylindrical shape, a central opening along a length of the bite block, and a pair of opposite open ends. An insertion slot is formed through the wall and along the entire length of the bite block. The bite block is positioned spaced vertically below the track and has a working end portion extending further rearward than a plane of the face contacting side of the track.

This Summary is provided only to summarize some example embodiments, so as to provide a basic understanding of some aspects of the subject matter described in this document. Accordingly, it will be appreciated that the features described in this Summary are only examples and should not be construed to narrow the scope or spirit of the subject matter described herein in any way. Unless otherwise stated, features described in the context of one example may be combined or used with features described in the context of one or more other examples. Other features, aspects, and advantages of the subject matter described herein will become apparent from the following Detailed Description, Figures, and Claims.

### BRIEF DESCRIPTION OF THE DRAWINGS

The discussion below makes reference to the following drawings, in which like reference characters may refer to like parts throughout, and in which:

FIG. 1 is a front, right, top perspective view of a system including a patient wearing a tube holding assembly;

FIG. 2 is a right side view of the patient of FIG. 1 wearing a portion of the tube holding assembly of FIG. 1;

FIG. 3 is a rear, left perspective view of the patient of FIGS. 1 and 2 wearing the tube holding assembly of FIGS. 1 and 2;

FIG. 4 is a front, right, top perspective view of the tube holding assembly of FIGS. 1-3;

FIG. 5 is a front, right, top perspective view of a portion of the tube holding assembly of FIGS. 1-4;

FIG. 6 is a front, right, top perspective view of a portion of the tube holding assembly of FIGS. 1-5;

FIG. 7 is a front, right, top perspective view of a mount subassembly of the tube holding assembly of FIGS. 1-6;

FIG. 8 is a front, right, top perspective view of a slider lock subassembly of the tube holding assembly of FIGS. 1-6;

FIG. 9 is a rear, left, top perspective view of the slider lock subassembly of FIGS. 1-6 and 8;

FIG. 10 is a front, right, top perspective view of the slider lock subassembly of FIGS. 1-6, 8, and 9, similar to FIG. 8 but with the slider lock subassembly closed;

FIG. 11 is a left, top perspective view of the slider lock subassembly of FIGS. 1-6 and 8-10, with the slider lock subassembly closed;

FIG. 12 is a front, right, top perspective view of a tube liner of the tube holding assembly of FIGS. 1-6;

FIG. 13A is a front, top perspective view of a tube stopper in isolation;

FIG. 13B is a front, top perspective view of the tube stopper of FIG. 13A and a tube positioned within a nostril of a patient;

FIG. 13C is a front, top perspective view of another tube stopper in isolation;

FIG. 13D is a front, top perspective view of the tube stopper of FIG. 13C and a tube positioned within a nostril of a patient;

FIG. 13E is a front, top perspective view of yet another tube stopper in isolation;

FIG. 13F is a front, top perspective view of the tube stopper of FIG. 13E and a tube positioned within a nostril of a patient;

FIG. 14A is a front, right, top perspective view of yet another tube stopper with a locking mechanism in an open configuration about a first tube;

FIG. 14B is a front, right, top perspective view of the tube stopper with locking mechanism of FIG. 14A in a closed configuration about the first tube;

FIG. 14C is a front, right, top perspective view of the tube stopper with locking mechanism of FIGS. 14A and 14B in a closed configuration about a second tube;

FIG. 14D is a front, right, top perspective view of yet another tube stopper with a locking mechanism in an open configuration about a tube;

FIG. 14E is a front, right, top perspective view of the tube stopper with locking mechanism of FIG. 14D in a closed configuration about the tube;

FIG. 15A is a front, right, top perspective view of yet another tube stopper with a locking mechanism about a tube and coupled to a mount of another tube holding assembly;

FIG. 15B is a rear, right, top perspective view of the tube holding assembly of FIG. 15A;

FIG. 15C is a top view of the tube holding assembly of FIGS. 15A and 15B, taken from line XVC-XVC of FIG. 15A;

FIG. 15D is a front, right, top perspective view of yet another tube stopper with a locking mechanism about a tube and coupled to a mount of another tube holding assembly;

FIG. 15E is a top view of the tube holding assembly of FIG. 15D, taken from line XVE-XVE of FIG. 15D;

FIG. 16 is a front, right, top perspective view of another system including the patient of FIGS. 1-3 wearing the tube holding assembly of FIGS. 15A-15C;

FIG. 17A is a rear, right, top perspective view of the patient of FIGS. 1-3 and 16 wearing the tube holding assembly of FIGS. 15A-15C and 16, with protective flaps open;

FIG. 17B is a rear, right, top perspective view of the patient of FIGS. 1-3, 16, and 17A wearing the tube holding assembly of FIGS. 15A-15C, 16, and 17A, with protective flaps closed;

FIGS. 18-20 are different perspective views of a system including a patient wearing a tube holding assembly of FIGS. 1-6 with multiple slider lock subassemblies of FIGS. 8-11;

FIG. 21 shows a front and side perspective view of one example of a device for holding or securing a tube (e.g., gastric (e.g., nasogastric or orogastric) or tracheal (e.g., endotracheal (e.g., nasotracheal or orotracheal)) tube) constructed in accordance with one or more embodiments of the disclosure and with a tube holder centered on the device;

FIG. 22 shows a perspective cross-section taken along line 2′-2′ of the device shown in FIG. 21;

FIG. 23 shows an alternate top, front, and side perspective view of the device shown in FIG. 21 and with the tube holder moved to a position offset from the centered position;

FIG. 24 shows a cross-section taken along line 4′-4′ of the device shown in FIG. 23;

FIG. 25 shows a rear and side perspective view of only the tube holder portion of the device shown in FIG. 21;

FIG. 26 shows a front view of the tube holder shown in FIG. 25;

FIG. 27 shows a rear view of the tube holder shown in FIG. 25;

FIG. 28 shows a right side view of the tube holder shown in FIG. 25;

FIG. 29 shows a top view of the tube holder shown in FIG. 25;

FIG. 30 shows a left side view of the tube holder of FIG. 25;

FIG. 31 shows a bottom view of the tube holder of FIG. 25;

FIG. 32 shows a rear and right perspective view of one alternative example of a tube holder for the device shown in FIG. 21;

FIG. 33 shows a rear view of the tube holder of FIG. 32;

FIG. 34 shows a rear and right perspective view of another alternative example of a tube holder for the device shown in FIG. 21;

FIG. 35 shows a rear view of the tube holder of FIG. 34;

FIG. 36 shows a rear and left side perspective view of another alternative example of a tube holder for the device shown in FIG. 21;

FIG. 37 shows a rear view of the tube holder of FIG. 36;

FIG. 38 shows a rear and left side perspective view of another alternative example of a tube holder for the device shown in FIG. 21;

FIG. 39 shows a rear view of the tube holder of FIG. 38;

FIG. 40 shows a rear and left side perspective view of another alternative example of a tube holder for the device shown in FIG. 21;

FIG. 41 shows a rear view of the tube holder of FIG. 40;

FIG. 42 shows a rear and left side perspective view of another alternative example of a tube holder for the device shown in FIG. 21;

FIG. 43 shows a rear view of the tube holder of FIG. 42;

FIG. 44 shows a rear and left side perspective view of another alternative example of a tube holder for the device shown in FIG. 21;

FIG. 45 shows a rear view of the tube holder of FIG. 44;

FIG. 46 shows a rear and left side perspective view of another alternative example of a tube holder for the device shown in FIG. 21;

FIG. 47 shows a rear view of the tube holder of FIG. 44;

FIG. 48 shows a rear and left side perspective view of another alternative example of a tube holder for the device shown in FIG. 21;

FIG. 49 shows a rear view of the tube holder of FIG. 48;

FIG. 50 shows a front and left side perspective view of the tube holder of FIG. 48 in use on a device like that in FIG. 21;

FIG. 51 shows a rear and left side perspective view of another alternative example of a tube holder for the device shown in FIG. 21;

FIG. 52 shows a rear view of the tube holder of FIG. 51;

FIG. 53 shows a front and left side perspective view of the tube holder of FIG. 51 in use on a device like that in FIG. 21;

FIG. 54 shows a rear and left side perspective view of another alternative example of a tube holder for the device shown in FIG. 21;

FIG. 55 shows a rear view of the tube holder of FIG. 54;

FIG. 56 shows a front and left side perspective view of the tube holder of FIG. 54 in use on a device like that in FIG. 21;

FIG. 57 shows a rear and left side perspective view of another alternative example of a tube holder for the device shown in FIG. 21;

FIG. 58 shows a rear view of the tube holder of FIG. 57;

FIG. 59 shows a front and left side perspective view of the tube holder of FIG. 57 in use on a device like that in FIG. 21; and

FIGS. 60-62 are different perspective views of a system including a patient wearing a device of FIGS. 21-24 and one or more slider lock subassemblies of FIGS. 8-11 with an optional tube holder of FIGS. 25-31 (not shown).

### DETAILED DESCRIPTION OF THE DISCLOSURE

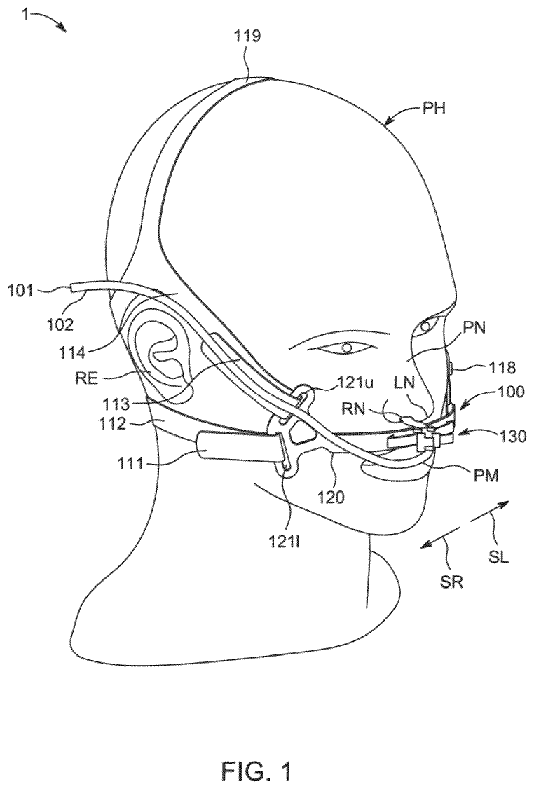

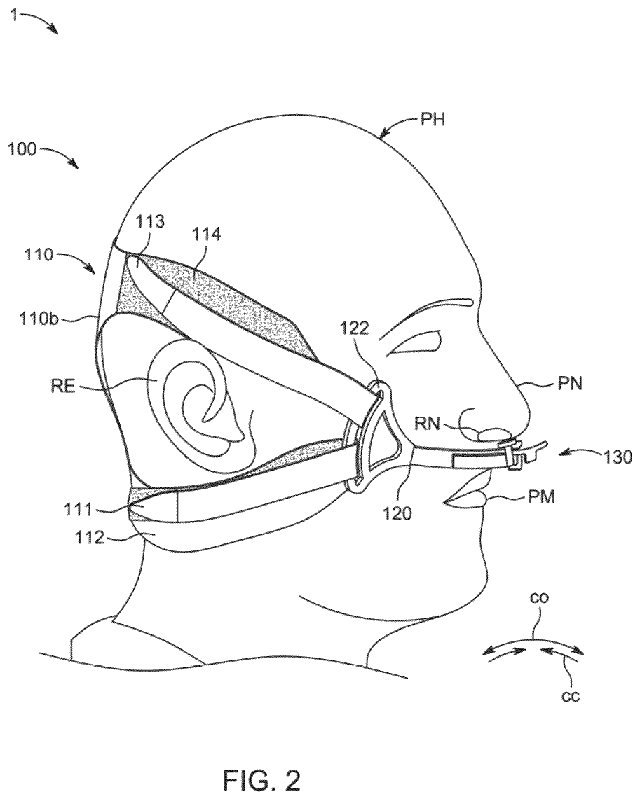

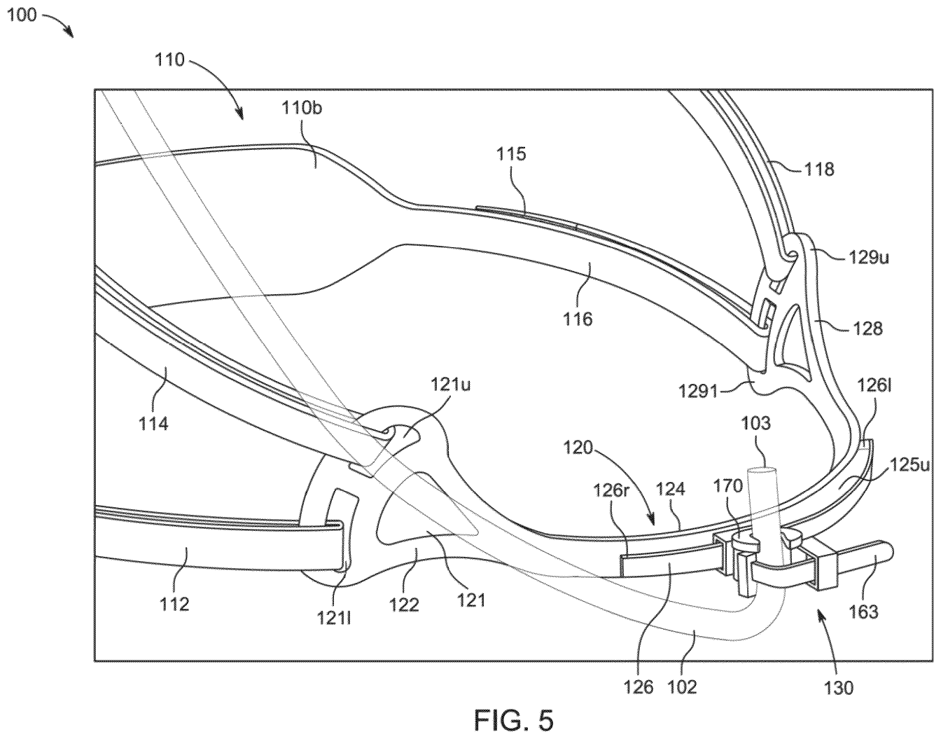

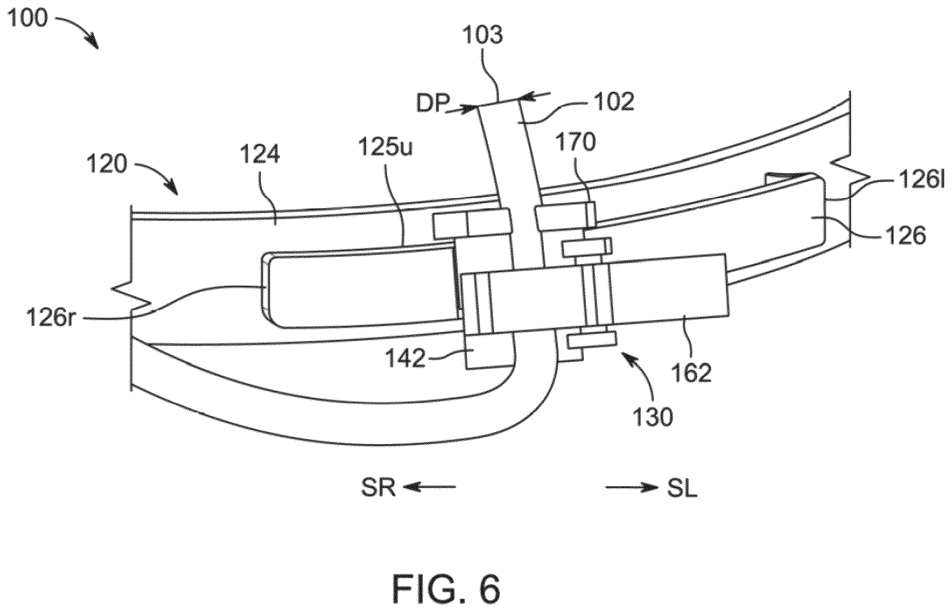

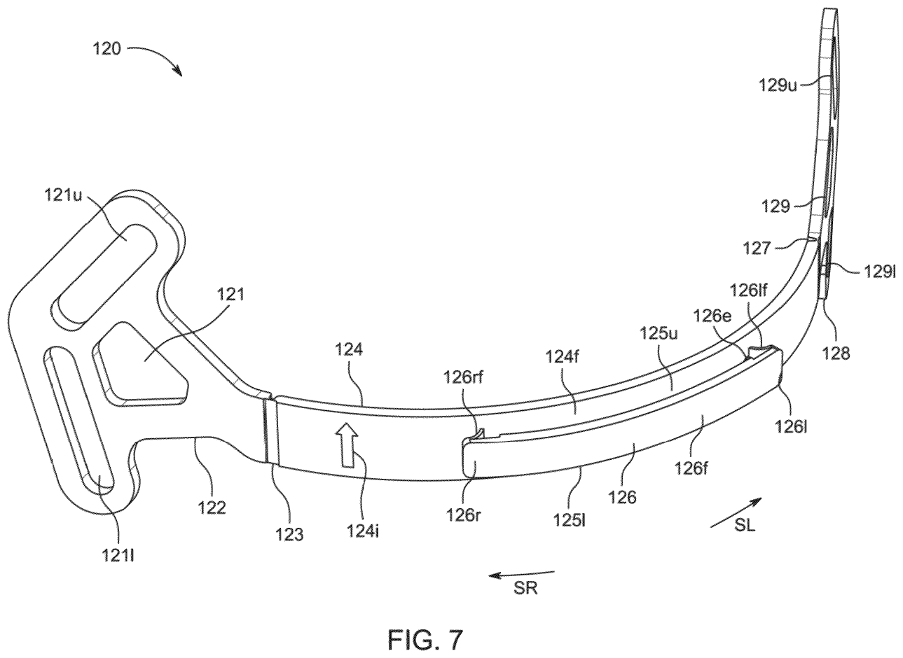

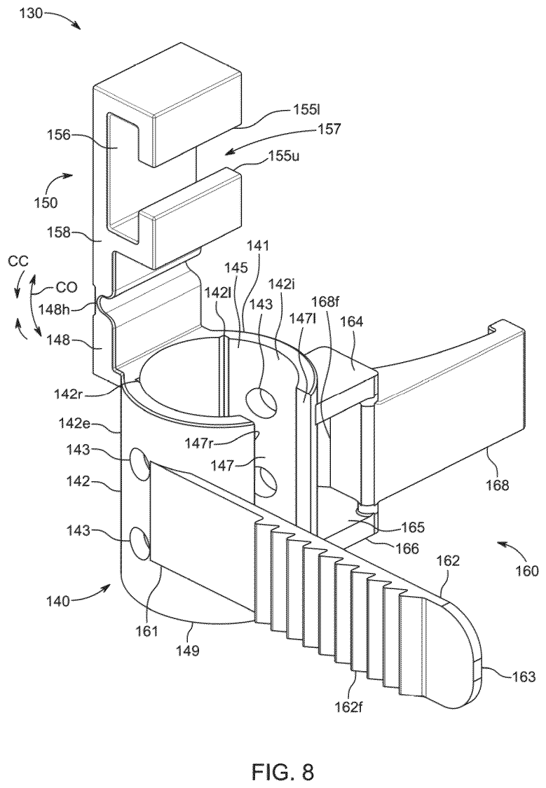

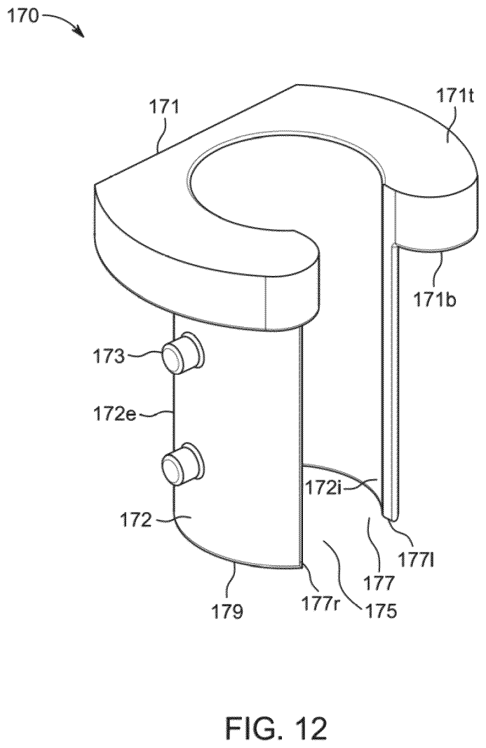

This application references prior filed U.S. Provisional Patent Application No. 62/689,386, filed Jun. 25, 2018, U.S. Pat. No. 11,241,367, issued Feb. 8, 2022, and U.S. Patent Application Publication No. 2014/0261462, published Sep. 18, 2014, each of which is hereby incorporated by reference herein in its entirety. FIGS. 1-3 show an illustrative system 1 including a tube holding assembly 100 that may be worn on a patient's head PH (e.g., a head with a right ear RE, a left ear LE, a mouth PM, a nose PN with a right nostril RN and/or a left nostril LN, and/or the like). Tube holding assembly 100, as shown in FIGS. 1-6 may include a tube 102, a strap subassembly 110, a mount subassembly 120 (e.g., as also shown in FIG. 7), a slider lock subassembly 130 (e.g., as also shown in FIGS. 8-11), and a tube liner 170 (e.g., as also shown in FIG. 12). Tube 102 may define a tube hollow passageway 102h, which may have an outer cross-sectional dimension (e.g., diameter) DP, extending between a proximal tube end 101 and a distal tube end 103. Tube 102 may be made of any suitable material(s), including polyurethane (“PU”), and may be introduced into a patient in any suitable manner for treating the patient in any suitable manner. Tube 102 may be any suitable tube, including, but not limited to, a gastric tube (e.g., a nasogastric tube or an orogastric tube), a tracheal tube (e.g., an endotracheal tube (e.g., a nasotracheal tube or an orotracheal tube)), and/or the like. As just one example, tube 102 may be used as a nasogastric (“NG”) tube, where distal tube end 103 may be inserted up through a patient's nostril (e.g., right nostril RN or left nostril LN of patient's head PH), then down through the patient's esophagus, and then into the patient's stomach. Once distal tube end 103 is in such a functional position or any other suitable position with respect to the patient (e.g., positioned in a particular target space within the patient), other portions of assembly 100 may be used to hold tube 102 in that position with respect to the patient and such that proximal tube end 101, and, thus, passageway 102h, may be accessible to an operator of assembly 100 (e.g., a physician or nurse or perhaps even the patient itself) for use in introducing any suitable material (e.g., food, air, medication, any other suitable fluids, and/or the like) into the target space within the patient via tube passageway 102h and/or for use in removing any suitable material (e.g., stomach contents, air, any other suitable fluids, and/or the like) from the target space within the patient via tube passageway 102h. Therefore, in some particular embodiments, assembly 100 may use tube 102 as a nasogastric tube for use in carrying out any suitable procedures via a nostril of the patient (e.g., feeding and/or intubation and/or specimen removal). Alternatively, assembly 100 may use tube 102 as any other suitable tube for use in carrying out any suitable procedures via any other suitable orifice of the patient (e.g., patient's mouth PM).

As shown in FIGS. 1-6, when assembly 100 is fully assembled for use in holding tube 102 in a functional position with respect to patient's head PH (e.g., extending into one of nostrils LN or RN), a portion of tube 102 may be held within tube liner 170, which may be held within slider lock subassembly 130, which may be coupled to mount subassembly 120, which may be coupled to strap subassembly 110, which may be coupled to patient's head PH. Strap subassembly 110 and mount subassembly 120, when coupled together, may be adjusted or manipulated in any suitable manner to fasten or secure or hold mount subassembly 120 in a particular position with respect to patient's head PH in a stable and/or tight and/or comfortable manner. Any suitable holder or strap subassembly 110 may be provided for coupling to and holding or securing or fastening any suitable mount subassembly 120 on patient's head PH (e.g., at a particular position with respect to head PH). For example, in some embodiments, as shown, strap subassembly 110 may include a strap base 110b from which may extend a lower right strap 112 to a lower right strap end 111, an upper right strap 114 to an upper right strap end 113, a lower left strap 116 to a lower left strap end 115, an upper left strap 118 to an upper left strap end 117, and/or a top strap 119, while mount subassembly 120 may include a mount base 124 extending between a right mount end 122 with at least one right mount end strap opening 121, and a left mount end 128 with at least one left mount end strap opening 129. An indicator 124i may be provided to indicate to an operator an intended orientation for use. As shown, for example, lower right strap end 111 of lower right strap 112 may be passed through a lower right mount end strap opening 1211 and pulled back towards and coupled to a portion of strap subassembly 110 (e.g., another portion of lower right strap 112) for coupling mount subassembly 120 to lower right strap 112, upper right strap end 113 of upper right strap 114 may be passed through an upper right mount end strap opening 121u and pulled back towards and coupled to a portion of strap subassembly 110 (e.g., another portion of upper right strap 114) for coupling mount subassembly 120 to upper right strap 114, lower left strap end 115 of lower left strap 116 may be passed through a lower left mount end strap opening 1291 and pulled back towards and coupled to a portion of strap subassembly 110 (e.g., another portion of lower left strap 116) for coupling mount subassembly 120 to lower left strap 116, and/or upper left strap end 117 of upper left strap 118 may be passed through an upper left mount end strap opening 129u and pulled back towards and coupled to a portion of strap subassembly 110 (e.g., another portion of upper left strap 118) for coupling mount subassembly 120 to upper left strap 118.

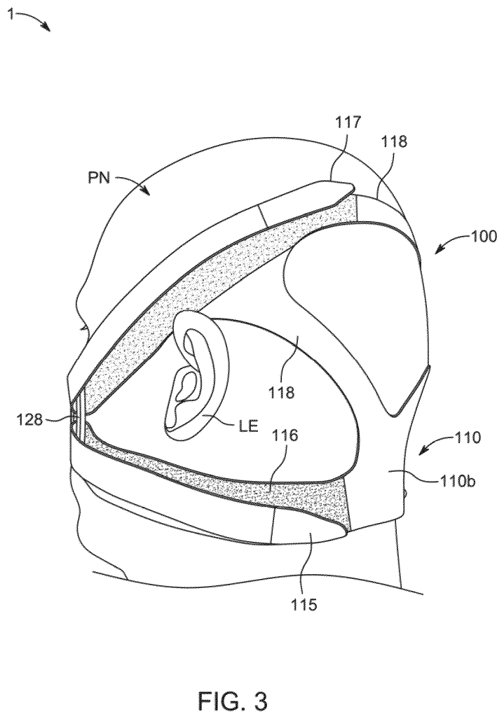

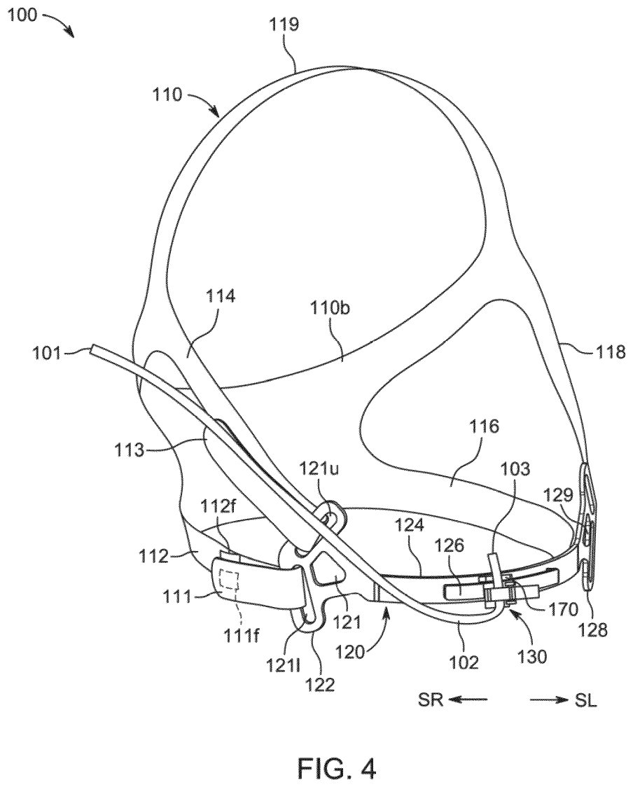

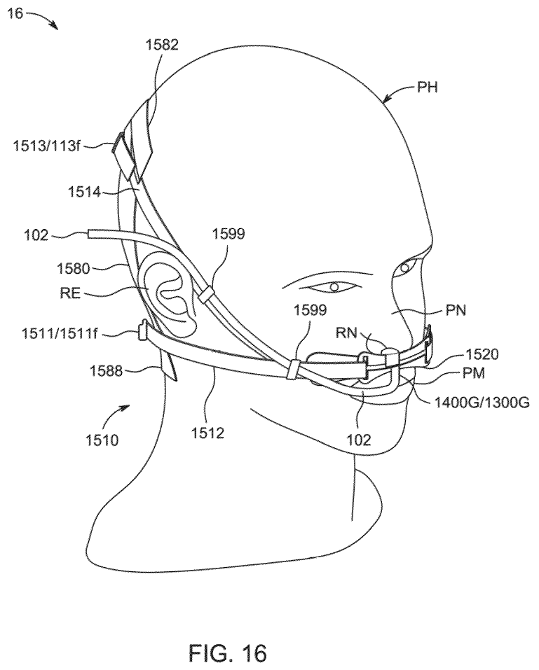

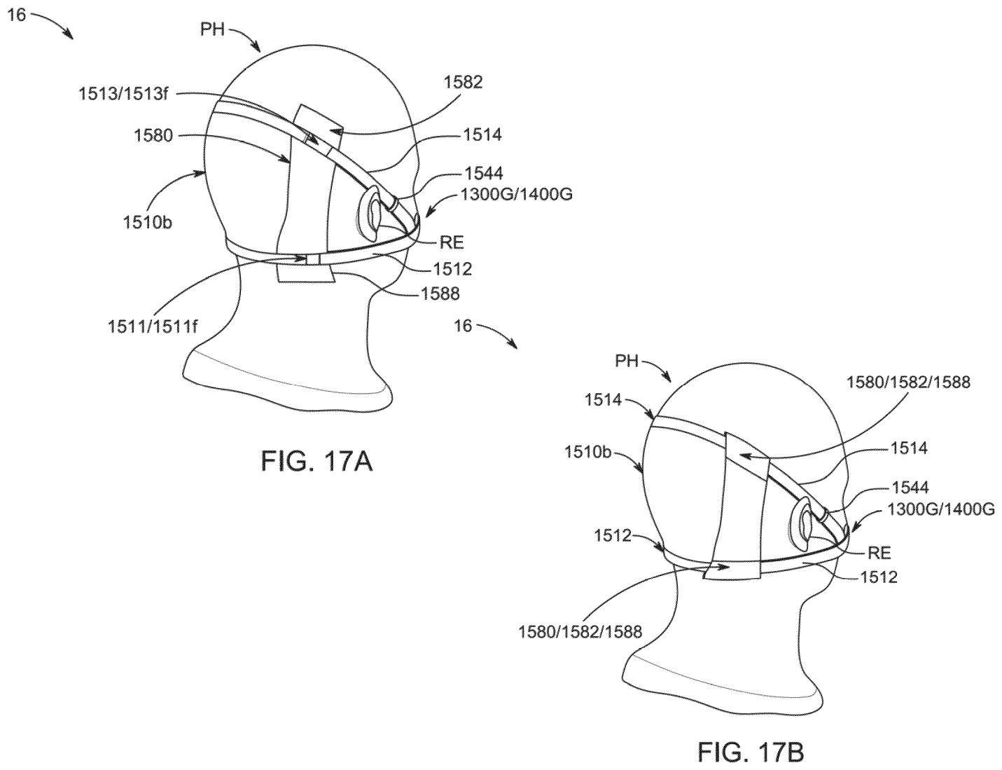

Each one of straps 112, 114, 116, and 118 may be provided with at least one set of coupling features for enabling the length of each strap to be adjusted. For example, as shown in FIG. 4, strap 112 may include at least one coupling feature 112f along at least a portion of a length of strap 112 while strap 112 may also include at least one other coupling feature 111f at or adjacent strap end 111, such that features 111f and 112f may be coupled to one another for maintaining a particular length of strap 112 to be extending between strap base 110b and mount subassembly 120 (e.g., opening 1211). For example, such coupling features 111f and 112f may be different but interlocking components of a hook and loop fastener assembly (e.g., Velcro™), thereby enabling strap end 111 to be removably coupled to another portion of strap 112 for easily adjusting the length of strap 112 extending between base 110b and mounting subassembly 120. Any other suitable coupling features may be used, including, but not limited to, snaps, clips, click fit strap connectors, a slide strap adjuster, any suitable adjuster buckle, and/or the like. In some embodiments, such as shown in FIGS. 16, 17A, and 17B with respect to a system 16 with another assembly, one or more protective flaps may be used to cover one, some, or each of the coupling features of one, some, or each strap, such that a patient may not easily access the coupling features for readjusting the fit of the assembly or for perhaps removing the assembly altogether.

The length of each one of straps 112, 114, 116, and 118 extending between strap base 110b and mount subassembly 120 may be independently adjusted based on the size and shape of patient's head PH (e.g., by varying the position at which a strap's free end is coupled to another portion of strap subassembly 110 after passing through an opening of mount subassembly 120), which may allow for tube holding assembly 100 to be securely yet comfortably worn on any suitably sized patient's head PH. When each one of straps 112, 114, 116, and 118 may be coupled to mount subassembly 120 in such a manner, mount base 124 may be securely yet comfortably held underneath patient's nose PN (e.g., above and along the upper lip of patient's mouth PM) while at least a portion of strap base 110b may be held against a portion of the back of patient's head PH, as lower right strap 112 may extend between base 110b and mount base 124 underneath the patient's right ear RE, as upper right strap 114 may extend between base 110b and mount base 124 above the patient's right ear RE, as lower left strap 116 may extend between base 110b and mount base 124 underneath the patient's left ear LE, as upper left strap 118 may extend between base 110b and mount base 124 above the patient's left ear LE, and/or as top strap 119 may extend over the top of patient's head PH.

Each one of straps 112, 114, 116, 118, 119, and/or base 110b may be made of any suitable material, including, but not limited to, nylon, cloth, mesh, foam, and/or the like, and may include any suitable comfort material provided on an inner surface of one or more of the straps or base that may interface with a portion of patient's head PH (e.g., a foam layer that may be adhered to an inner surface of each strap for providing a comfortable interface between strap subassembly 110 and patient's head PH when tube holding assembly 100 is held in an appropriate position on patient's head PH). A length of top strap 119 may be adjustable using any suitable mechanism (e.g., similar to one of the other straps, but through an opening in one of the upper straps or around an upper strap) or may be made of an elastic material in order to stretch or contract for making appropriate contact with a top of patient's head PH. In other embodiments, lower right strap 112 and upper right strap 114 may each extend from different portions of strap base 110b to a single right strap end (not shown) that may be passed through a single right mount end strap opening 121 for enabling adjustment with respect to mount subassembly 120, while lower left strap 116 and upper left strap 118 may each extend from different portions of strap base 110b to a single left strap end (not shown) that may be passed through a single left mount end strap opening 129 for enabling adjustment with respect to mount subassembly 120. In yet other embodiments, strap base 110b may not exist, and a lower strap may extend between lower right strap end 111 and lower left strap end 115, while an upper strap, distinct from such a lower strap, may extend between upper right strap end 113 and upper left strap end 117, while top strap 119 may extend between different portions of such an upper strap, or may not be provided at all. Any other suitable additional or alternative mechanisms may be provided by strap subassembly 110 or otherwise for holding mount subassembly 120 in a particular position with respect to patient's head PH.

Mount subassembly 120 may include one or more hinge elements (e.g., one or more living hinges) along its length for enabling mount subassembly to flex or curve along a portion of patient's head PH. For example, as shown, a right mount hinge 123 may be provided between mount base 124 and right mount end 122, and/or a left mount hinge 127 may be provided between mount base 124 and left mount end 128, where such hinge(s) may enable the shape of mount subassembly 120 between ends 122 and 128 bend or flex to suit different heads of different shapes and sizes (e.g., hinge 123 may enable right mount end 122 to form any suitable angle with respect to an adjacent portion of mount base 124 such that end 122 may extend along a portion of a side of a patient's face while base 124 may extend along a portion of a front of a patient's face). Mount subassembly 120 may also include a slider clamp mount track body 126 that may extend between a slider clamp mount track left end 1261 and a slider clamp mount track right end 126r. Track body 126 may include an extension ledge 126e extending forwardly out from a front face 124f of mount base 124, while a front wall 126f of track body 126 may extend upwardly and/or downwardly (e.g., perpendicularly) from a front of extension ledge 126e, while a right lock 126rf (e.g., flexible flap) may extend backward from front wall 126f at right end 126r towards front face 124f of mount base 124, and/or while a left lock 126lf (e.g., flexible flap) may extend backward from front wall 126f at left end 1261 towards front face 124f of mount base 124. Such a structure may create a slider clamp mount track space, such as a lower slider clamp mount track space 1251 between front face 124f of mount base 124 and a rear surface of front wall 126f below extension ledge 126e as extending between locks 126rf and 126lf, and/or such as an upper slider clamp mount track space 125u between front face 124f of mount base 124 and a rear surface of front wall 126f above extension ledge 126e as extending between locks 126rf and 126lf. One or both of such locks 126rf and 126lf may be flexible for enabling access to one or both of track spaces 125u and 1251 by one or more features of a slider clamp 150 of slider lock subassembly 130 (e.g., one or both of upper slider clamp feature 155u and lower slider clamp feature 1551, respectively, of clamp 150), such that slider lock subassembly 130 (e.g., when holding or about to hold tube 102) may be able to be held close to but also slide along mount base 124 (e.g., in the direction of slide left arrow SL of the track and/or in the direction of slide right arrow SR of the track). A rear face opposite front face 124f of mount base 124 may be operative to interface with (e.g., be held against) a portion of patient's head PH (e.g., above and/or along an upper lip of patient's mouth PM and/or just below and/or adjacent patient's nose PN).

Mount subassembly 120 may be made of any suitable material(s) through any suitable procedures, including, but not limited to, molded plastic, metal, and/or the like. At least a portion of mount base 124 may be provided by a material with enough rigidity to provide a mount track space(s) that may allow slider lock assembly 130 to slide therein for aligning with any suitable feature(s) of the patient (e.g., with a particular one of nostrils RN and LN) and/or to enable strap subassembly 110 to hold mount subassembly 120 in a particular position with respect to patient's head PH. Additionally, in some embodiments, mount subassembly 120 may include any suitable comfort material provided on a surface of mount base 124 that may interface with a portion of patient's head PH (e.g., a cushioning foam layer that may be adhered to mount base 124 (e.g., along the face opposite front face 124f) for providing a comfortable interface between mount base 124 and patient's head PH (e.g., the upper lip of patient's mouth PM) when tube holding assembly 100 is held in an appropriate position on patient's head PH). Mount base 124 may be any suitable size (e.g., 19 centimeters in length) for use with any suitable size patient. Different size assembly components may be used for different size patients.

Slider lock subassembly 130 may include a tube clamp 140, a slider clamp 150, and a tube adjuster 160. Tube clamp 140 may include a tube clamp body 142 of any suitable shape. For example, as shown, tube clamp body 142 may have a substantially hollow cylindrical shape, with a tube clamp body exterior wall surface 142e and a tube clamp body interior wall surface 142i extending between a tube clamp body top 141 and a tube clamp body bottom 149 for defining a hollow tube clamp body passageway 145 that may be accessible via top 141 and/or bottom 149, as well as via a tube clamp body opening 147 that may extend along a length of body 142 between a tube clamp body left edge 1471 and a tube clamp body right edge 147r. One or more hinges, such as a tube clamp body left hinge 1421 and/or a tube clamp body right hinge 142r, may be provided along the length of body 142 (e.g., along a length of tube clamp body interior wall surface 142i) for enabling the width of tube clamp body opening 147 between edges 1471 and 147r to expand or contract, thereby expanding or contracting a cross-sectional area or any suitable cross-sectional dimension(s) of hollow tube clamp body passageway 145, which may allow any suitable component(s) (e.g., tube 102 with or without tube liner 170) to be loosely positioned therein and then clamped tightly therein.

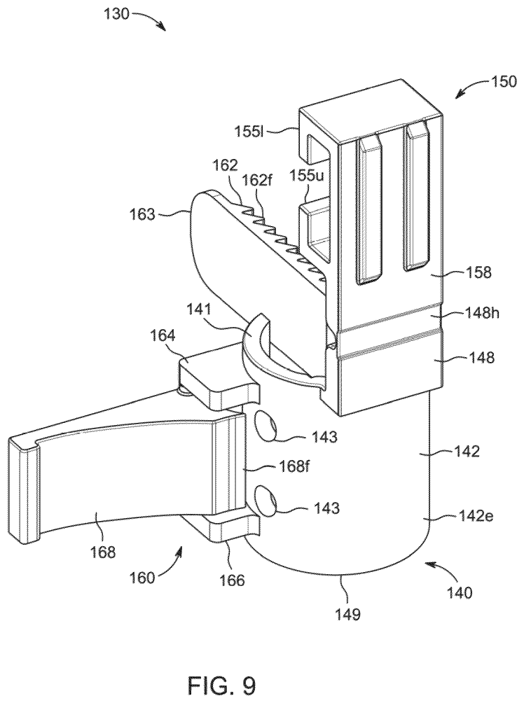

Tube adjuster 160 may be coupled to tube clamp 140 for selectively expanding or contracting or maintaining a cross-sectional area or any suitable cross-sectional dimension(s) of hollow tube clamp body passageway 145. For example, as shown, tube adjuster 160 may include a tube adjuster tie 162 extending between a tube adjuster tie fixed end 161, which may be coupled to a portion of exterior wall surface 142e of tube clamp body 142 adjacent tube clamp body right edge 147r, and a tube adjuster tie free end 163. Additionally, as shown, tube adjuster 160 may include a pawl 168 rotatably or otherwise movably coupled between a pawl top 164 and a pawl bottom 166, each of which may be coupled to a portion of exterior wall surface 142e of tube clamp body 142 adjacent tube clamp body left edge 1471, such that at least one pawl feature 168f of pawl 168 may be positioned within a pawl opening 165 that may be defined between pawl 168 and pawl top 164 and pawl bottom 166 and tube clamp body 142. Tube adjuster 160 may be manipulated (e.g., by any suitable operator of assembly 100) between an open configuration of FIGS. 8 and 9 and a closed configuration of FIGS. 1-6, 10, and 11 by passing tube adjuster tie free end 163 across and beyond tube clamp body opening 147 and into pawl opening 165, whereby any suitable tube adjuster tie feature(s) 162f that may be provided along a surface of tube adjuster tie 162 may be engaged by any suitable pawl feature(s) 168f for holding tube adjuster tie 162 at a particular position within and with respect to pawl opening 165 and pawl feature(s) 168f. The further tube adjuster tie 162 may be inserted into opening 165, the more a cross-sectional dimension (e.g., periphery) of hollow tube clamp body passageway 145 may contract. Tube adjuster tie feature(s) 162f and pawl feature(s) 168f may interact to engage and retain tube adjuster tie 162 in a particular position within opening 165 and may be configured only to let tie 162 be further inserted into opening 165 but not to let tie 162 be removed from opening 165 unless pawl 168 is used to release the engagement of pawl feature(s) 168f from tube adjuster tie feature(s) 162f. Therefore, an assembly operator may be enabled to ratchet tie 162 further into and through opening 165 for tightening a clamping of any suitable component(s) within tube clamp body passageway 145. Any suitable mechanisms in addition to or as an alternative to a ratchet and pawl mechanism may be used to enable such controlled contracting and expanding and maintaining of the dimension(s) of tube clamp body passageway 145.

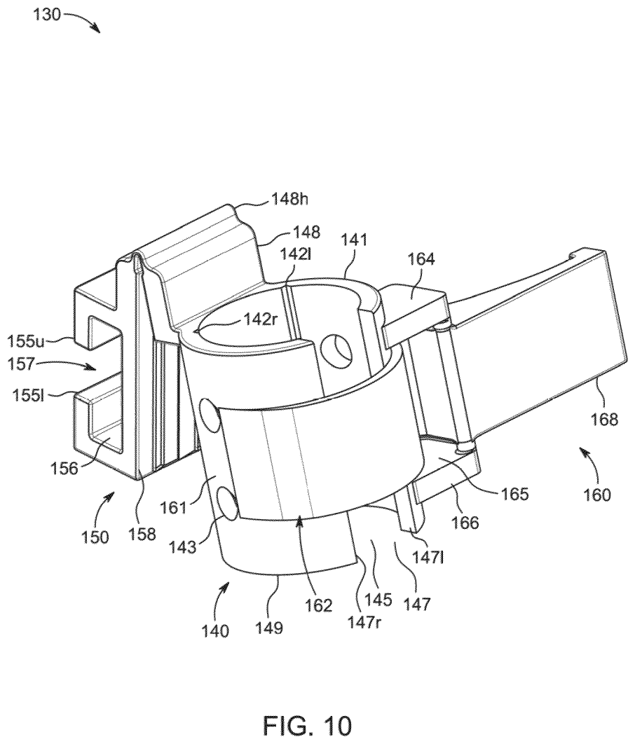

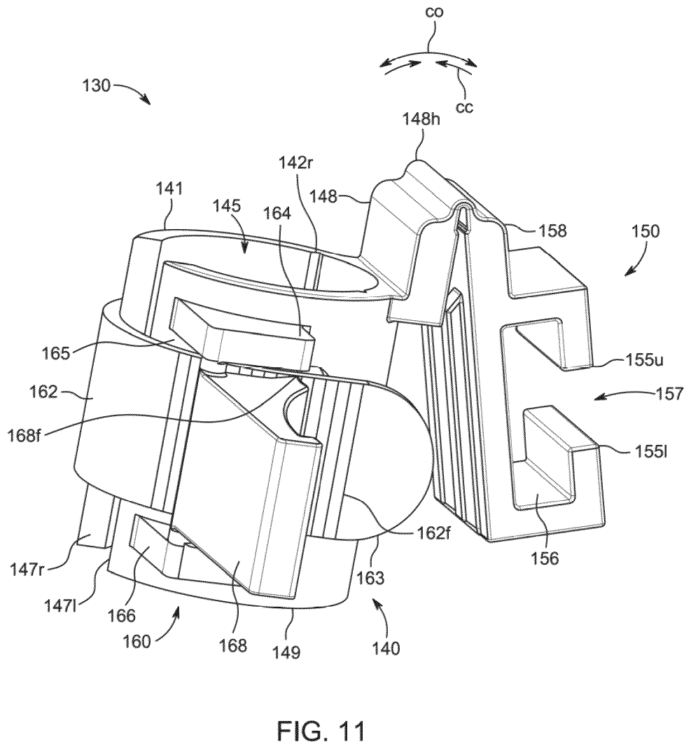

Slider clamp 150 may include a slider clamp body 158 that may be any suitable shape for defining any suitable slider clamp body passageway 156 of any suitable shape. For example, as shown, slider clamp body 158 may define a T-shaped slider clamp body passageway 156 extending through a slider clamp body opening 157 between a lower slider clamp feature 1551 of slider clamp body 158 and an upper slider clamp feature 155u of slider clamp body 158. Slider clamp 150 may be coupled to a tube clamp body slider extension 148 of tube clamp 140 (e.g., as may be coupled to tube clamp body 142 adjacent tube clamp body top 141), such as via a tube clamp body slider extension hinge 148h, for enabling slider clamp 150 to be moved (e.g., rotated, bent, etc.) with respect to tube clamp 140 (e.g., about hinge 148h) between an open configuration of FIGS. 8 and 9 (e.g., forming at least a 180 degree angle or possibly greater angle between extension 148 and body 158) and a closed configuration of FIGS. 10, and 11 (e.g., forming no more than a 5-10 degree angle or possibly smaller angle (e.g., 0 degree angle) between extension 148 and body 158) or anywhere in between (e.g., in the direction of clamp close arrows CC or in the direction of clamp open arrows CO). Hinge 148h may be any suitable mechanical bearing that may allow any suitable movement between clamp body 142 and clamp body 158 (e.g., an ideal hinge or any other suitable hinge) that may be made of one or more suitable flexible materials and/or any suitable moving components. As just one example, hinge 148h may be a living hinge. As shown, at least one of locks 126rf and 126lf of mount subassembly 120 may be flexible for enabling access by one or each of upper slider clamp feature 155u and lower slider clamp feature 1551 of clamp 150 to a respective one of track spaces 125u and 125l. This may enable slider clamp 150 to be held within track spaces 125 and between locks 126rf and 126lf of mount subassembly 120 such that slider lock subassembly 130 (e.g., when in a closed position of FIGS. 1-6, 10, and 11 (e.g., when holding or about to hold tube 102)) may be held close to mount base 124 but also allowed to slide along mount base 124 (e.g., in the direction of slide left arrow SL and/or in the direction of slide right arrow SR). It is to be understood that the slide directions SL and SR of slider clamp body 158 with respect to the track of mount subassembly 120 may be axially opposite each other or opposite each other along a curved track (e.g., as shown) such that the directions of arrows SL and SR may be linearly opposite each other at any given point of body 158 along a curved track but the directions may be curved along the length of the track.

Tube liner 170 may include a tube liner body 172 of any suitable shape. For example, as shown, tube liner body 172 may have a substantially hollow cylindrical shape, with a tube liner body exterior wall surface 172e and a tube liner body interior wall surface 172i extending between a tube liner body top 171 and a tube liner body bottom 179 for defining a hollow tube liner body passageway 175 that may be accessible via top 171 and/or bottom 179, as well as via a tube liner body opening 177 that may extend along a length of body 172 between a tube liner body left edge 1771 and a tube liner body right edge 177r. Tube liner body 172 may be made of any suitable material, such as silicone or foam and/or the like, or may be provided with one or more hinges, for enabling the width of tube liner body opening 177 between edges 1771 and 177r to expand or contract, thereby expanding or contracting a cross-sectional area or any suitable cross-sectional dimension(s) of hollow tube liner body passageway 175, which may allow any suitable component(s) (e.g., tube 102) to be loosely positioned therein and then clamped tightly therein. Therefore, tube liner body 172 may be configured to function similarly to tube clamp body 142, but may be provided to provide a more soft or forgiving interface with tube 102 (e.g., to prevent tearing or kinking or pinching material of tube 102 and/or to prevent slider lock subassembly 130 from limiting the size of tube hollow 102h such that fluid may flow freely therethrough) than may be provided by tube clamp body 142. When tube adjuster 160 is used to tighten (e.g., ratchet) tube clamp 140, liner body 172 may more tightly grab onto and/or squeeze onto and/or gently but more firmly hold tube 102. Alternatively, tube liner body 172 may not be needed and tube 102 may be positioned within and clamped directly by tube clamp body 142. However, if used, as shown, tube liner body 172 may include one or more tube liner body clamp features 173 that may extend outwardly from tube liner body exterior wall surface 172e of tube liner body 172 and that may be operative to engage or otherwise interact with one or more respective tube clamp body liner features 143 that may extend inwardly into tube clamp body interior wall surface 142i of tube clamp body 142, such that tube liner body 172 may not move with respect to tube clamp body 142 (e.g., out from either the top or bottom of tube clamp body 142 (e.g., when a tugging force is applied to tube 102)). Moreover, as shown, tube liner body top 171 of tube liner 170 may be larger than (e.g., extend upwardly and outwardly from) tube liner body 172 between a top surface 171t and a bottom surface 171b. Bottom surface 171b may be operative to rest against tube clamp body top 141 of tube clamp body 142, while top surface 171t may be operative to support or interface with a bottom of patient's nose PN when assembly 100 is being worn by patient's head PH. With or without tube liner 170, tube adjuster 160 and tube clamp 140 may be configured to clamp and hold different tubes with different tube hollows of different cross-sectional dimensions (e.g., tube 102 with hollow 102h of dimension DP and/or tube 102* with hollow 102h* of dimension DP′ that may be greater than DP by any suitable magnitude (e.g., 18 Fr. (e.g. 6.0 millimeters) versus 16 Fr. (e.g. 5.3 millimeters) versus 14 Fr. (e.g. 4.7 millimeters) versus 12 Fr. (e.g. 4.0 millimeters) versus 10 Fr. (e.g. 3.2 millimeters) or the like)).

Therefore, as shown, when worn by patient's head PH, slider lock subassembly 130, with or without tube liner 170, may clamp a portion of tube 102 securely within passageway 145 of tube clamp 140 of slider lock subassembly 130 without limiting fluid flow through tube hollow 102h of that tube portion, while slider clamp 150 may movably couple slider lock subassembly 130 to mount subassembly 120 (e.g., such that slider lock subassembly 130 may be free to slide at least slightly in the direction of each one of slide left arrow SL and slide right arrow SR with respect to slider clamp mount track body 126 when slider clamp 150 is mated with slider clamp mount track body 126 within spaces 1251 and 125u), and while strap subassembly 110 may hold mount subassembly 120 in a particular position with respect to patient's head PH. Therefore, when properly worn, assembly 100 may enable certain sliding movement of subassembly 130 and tube 102 with respect to mount subassembly 120 (e.g., in the direction of arrow SL and/or in the direction of arrow SR along track body 126) and/or certain rotational movement of tube clamp 140 and tube 102 with respect to mount subassembly 120 (e.g., in the direction of arrow CC and/or in the direction of arrow CO about hinge 148h), while tube 102 may be fixed to tube clamp 140 and while mount subassembly 120 may be substantially fixed with respect to patient's head PH.

Therefore, when tube 102 may be used as a nasogastric tube and may be introduced into the patient via a nostril, assembly 100 may be used to prevent proximal or distal migration of tube 102 with respect to the patient once inserted into the patient. Assembly 100 may also be used to prevent the patient from pulling tube 102 out from within the patient (e.g., intentionally or accidentally). Assembly 100 may be used to maintain a position of tube 102 after insertion of tube 102 into the patient, which may be particularly helpful for confused and/or aggressive and/or combative patients that may attempt to dislodge the tube. By maintaining the proper position of the tube within the patient, such as by maintaining distal tube end 103 at a proper distance within the patient's stomach may reduce risk of aspiration related to proximal migration of the tube or coiling in the esophagus. For example, the tube may be inserted into the patient, such as via a nostril, then the functional position of the tube (e.g., the distal tube end) inside the patient may be verified (e.g., via X-ray to ensure the distal tube end is in the stomach), and then the remainder of assembly 100 (e.g., subassemblies 110, 120, and 130, with or without liner 170) may be implemented with respect to that properly positioned tube for securely maintaining that proper tube position with respect to the patient's head.

As one example of use of assembly 100, once tube 102 has been properly positioned within the patient, such as through left nostril LN, an operator may attach one side of the straps of strap subassembly 110 to one side of mount subassembly 120, if they haven't been already, such as by looping lower right strap end 111 of lower right strap 112 through lower right opening 1211 of mount base 124 and by looping upper right strap end 113 of upper right strap 114 through upper right opening 121u of mount base 124. Then, the operator may movably couple slider clamp 150 to slider clamp mount track body 126 of mount subassembly 120, if it is not yet already. Then the operator may position mount base 124 underneath patient's nose PN, such as along and above the upper lip of patient's mount PM, and then the operator may slide slider lock subassembly 130 along mount subassembly 120 such that tube clamp 140 is underneath the nostril into which tube 102 is entering the patient. Tube clamp 140, with or without liner 170 therein, may be rotated up (e.g., in the direction of arrow CO about hinge 148h) with respect to slider clamp 150 and mount subassembly 120 in order to press a portion of tube 102 more easily into passageway 145 of tube clamp body 142 via opening 147 and into passageway 175 of tube liner body 172 via opening 177, if provided. Then, once tube 102 has been positioned to extend through tube clamp body 142 of slider lock subassembly 130, whether or not also through any tube liner 170, the operator may use (e.g., ratchet) tube adjuster 160 to contract tube clamp body 142 to clamp and hold tube 102 with respect to tube clamp body 142 (e.g., such that the portion of tube 102 extending within passageway 145 between bottom 149 and top 141 may not be pulled downwardly out from passageway 145 beyond bottom 149 and/or may not be pulled upwardly out from passageway 145 beyond top 141 and/or may not be pulled outwardly from passageway 145 through opening 147). The operator may tighten or loosen tie 162 with respect to pawl 168 in order to stabilize tube 102. The operator may pull on tube 102, such as the portion extending downwardly out from passageway 145 below bottom 149, to ensure that tube 102 does not slide with respect to tube clamp 140. Then, the operator may wrap a portion of strap subassembly 110 behind patient's head PH in order to loop end 115 of lower left strap 116 below left ear LE and through lower left opening 1291 of mount base 124 and by looping end 117 of upper left strap 118 above left ear LE and through upper left opening 129u of mount base 124. Then, the operator may selectively adjust the length of one or more of straps 112, 114, 116, and 118 extending between strap base 110b and mount subassembly 120 (e.g., by varying the position at which a strap's free end is coupled to another portion of strap subassembly 110 after passing through an opening of mount subassembly 120), which may allow for tube holding assembly 100 to be securely yet comfortably worn on any suitably sized patient's head PH and such that mount base 124 may be securely yet comfortably held underneath patient's nose PN (e.g., above and along the upper lip of patient's mouth PM) while at least a portion of strap subassembly 110 (e.g., base 110b) may be held against a portion of the back of patient's head PH (e.g., while lower right strap 112 may extend between base 110b and mount base 124 underneath the patient's right ear RE, while upper right strap 114 may extend between base 110b and mount base 124 above the patient's right ear RE, while lower left strap 116 may extend between base 110b and mount base 124 underneath the patient's left ear LE, while upper left strap 118 may extend between base 110b and mount base 124 above the patient's left ear LE, and/or while top strap 119 may extend over the top of patient's head PH). The operator may ensure that the back of strap subassembly 110 (e.g., base 110b) is centered at the back of patient's head PH and, if not, may loosen or tighten any of the straps from either side to correct this. Alternatively, once tube 102 is properly positioned with respect to the patient (e.g., extending into a nostril) and once slider lock subassembly 130 has been coupled to mount subassembly 120, the operator may fully assemble strap subassembly 110 to mount subassembly 120 and attach strap subassembly 110 and mount subassembly 120 to patient's head PH (e.g., such that mount base 124 may extend along and above patient's mouth PM). Then, the operator may move (e.g., slide) slider lock subassembly 130 along mount subassembly 120 horizontally (e.g., in the direction of arrow SL or arrow SR) such that tube clamp 140 is properly aligned with tube 102 (e.g., underneath a nostril into which the tube extends) and/or may move (e.g., rotate) tube clamp 140 with respect to slider clamp 150 and mount subassembly 120 (e.g., about hinge 148h (e.g., in the direction of arrow CC or arrow CO)) such that tube clamp opening 147 of tube clamp body 142 is properly aligned with tube 102 for making easier the insertion of tube 102 through opening 147 into passageway 145. Then, tube 102 may be clamped and held within tube clamp body 142, and tube clamp 140 may be allowed to freely rotate about hinge 148h with respect to slider clamp 150 and mount subassembly 120 and freely slide with respect to mount subassembly 120 along track body 126 while tube 102 may remain properly positioned with respect to the patient despite such limited freedom of movement of tube clamp 140 with respect to mount subassembly 120. As shown, tube clamp passageway 145 may generally extend perpendicular to each one of arrows SL and SR when subassembly 130 is coupled to subassembly 120, no matter how body 142 may be positioned with respect to body 158 using hinge 148h (e.g., hinge 148h may enable body 142 to rotate with respect to body 158 about an axis of rotation that may be parallel to and/or linear with arrow SL and/or arrow SR (e.g., when body 158 is at a particular position along the track (e.g., even when the track may be curved)) and/or that may be perpendicular to an axis of passageway 145 along which body 142 may hold a portion of tube 102).

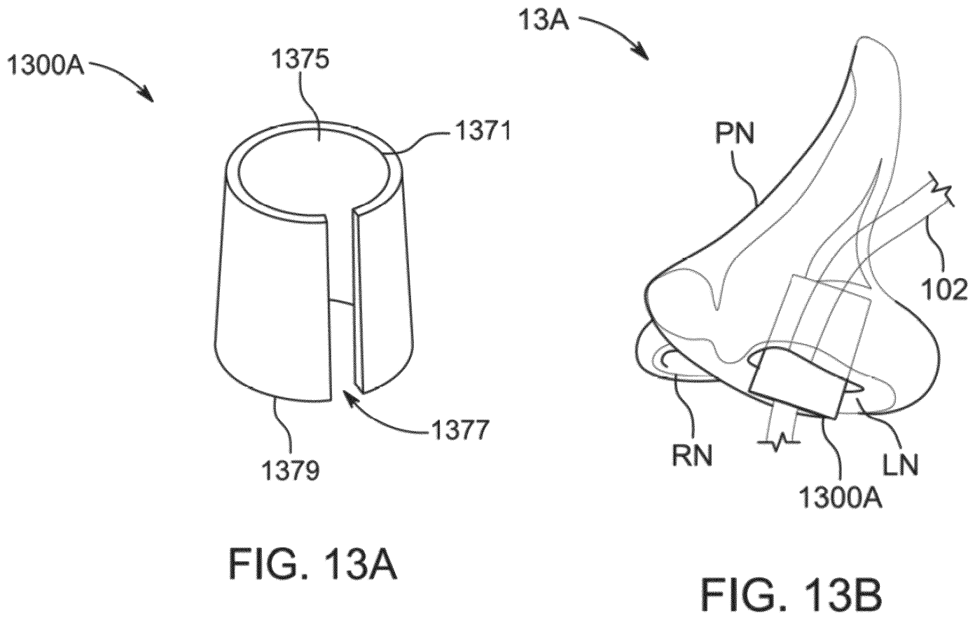

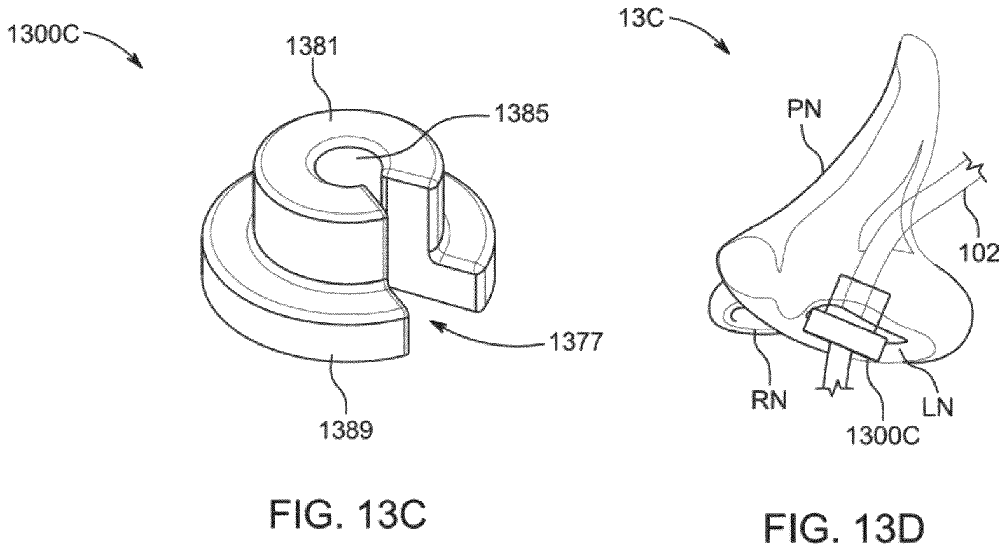

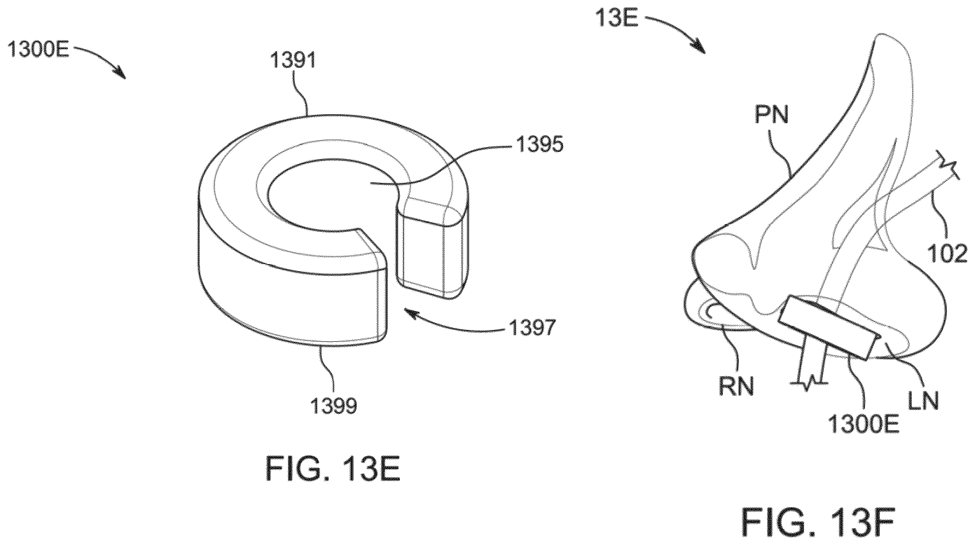

In some embodiments, in addition to or as an alternative to any of the components of assembly 100, a tube stopper may be provided about a portion of a tube for helping to hold the portion of the tube within a nostril of a patient. For example, as shown in FIG. 13A, a tube stopper 1300A may be a hollow conical shaped stopper extending between a top end 1371 and a bottom end 1379 that may be larger than top end 1371 through which a passageway 1375 may extend and may be exposed by an opening 1377, similar to liner 170. This may enable a portion of tube 102 to be passed (e.g., pressed) through opening 1377 and into passageway 1375 and such that at least top end 1371 may then be inserted into nostril LN, which may center tube 102 within the nostril or otherwise remove tube 102 from rubbing against an inner surface of the nostril, as shown by system 13A of FIG. 13B. Stopper 1300A may be configured to easily fit into and/or grip any suitable nostril and/or to grip and prevent slippage of any tube extending therethrough. As another example, as shown in FIG. 13C. a tube stopper 1300C may be a hollow stepped ring stopper extending between a top cylindrical end 1381 and a bottom cylindrical end 1389 that may be larger than top cylindrical end 1381 through which a passageway 1385 may extend and may be exposed by an opening 1387. This may enable a portion of tube 102 to be passed (e.g., pressed) through opening 1387 and into passageway 1385 and such that at least top cylindrical end 1381 may then be inserted into nostril LN, which may center tube 102 within the nostril or otherwise remove tube 102 from rubbing against an inner surface of the nostril, as shown by system 13C of FIG. 13D. Top cylindrical end 1381 of stopper 1300C may be configured to easily fit into and/or grip any suitable nostril and/or to grip and prevent slippage of any tube extending therethrough, while stepped bottom cylindrical end 1389 may prevent stopper 1300C and tube 102 from being pulled further into the nostril and/or prevent stopper 1300C and tube 102 from getting dislocated sideways. As yet another example, as shown in FIG. 13E. a tube stopper 1300E may be a hollow flanged stopper extending between a top cylindrical end 1391 and a bottom cylindrical end 1399 through which a passageway 1395 may extend and may be exposed by an opening 1397. This may enable a portion of tube 102 to be passed (e.g., pressed) through opening 1397 and into passageway 1395 and such that at least top cylindrical end 1391 may then be inserted into nostril LN, which may center tube 102 within the nostril or otherwise remove tube 102 from rubbing against an inner surface of the nostril, as shown by system 13E of FIG. 13F. Top cylindrical end 1391 of stopper 1300E may be configured to easily fit into and/or grip any suitable nostril and/or to grip and prevent slippage of any tube extending therethrough, while bottom cylindrical end 1399 may prevent stopper 1300E and tube 102 from being pulled further into the nostril.

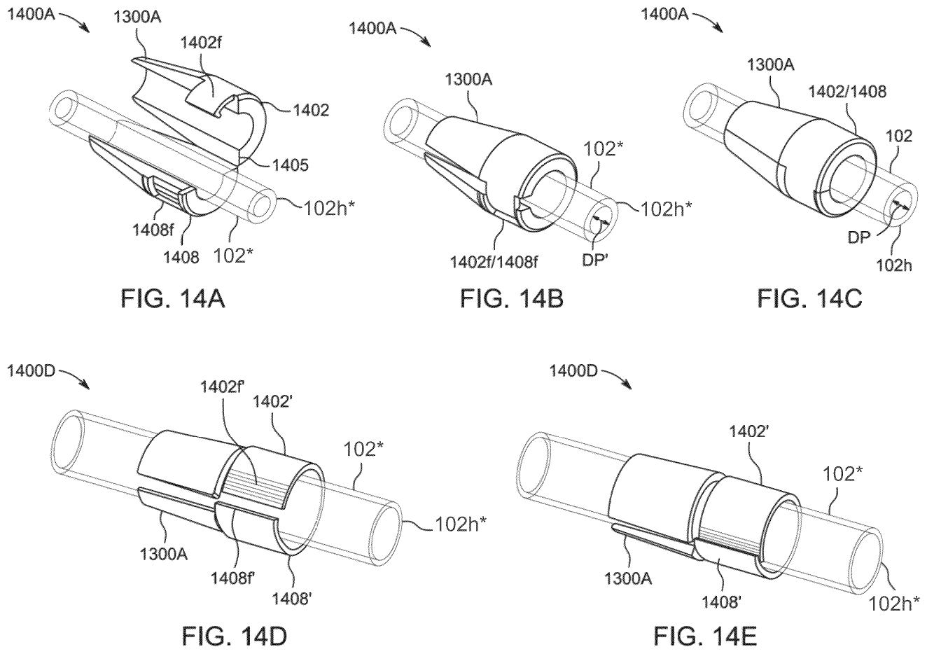

Any suitable stopper may be coupled to or integrated with any suitable clip or clamp for more securely fixing the stopper to a tube and/or for enabling the stopper to be coupled to a mount subassembly of a strapped tube holding assembly. For example, as shown in FIGS. 14A-14C, the bottom end of stopper 1300A (e.g., bottom end 1379) may be coupled to (e.g., adhered to) or integrated with (e.g., molded (e.g., double shot molded) with) a top end of a locking mechanism or hinged clip 1400A that may include a first portion 1402 and a second portion 1408 rotatably coupled about a hinge portion 1405 for either exposing passageway 1375 of stopper 1300A and clip 1400A for enabling tube 102 to be positioned therein or for closing clip 1400A about tube 102. As shown, portion 1402 may include any suitable feature(s) 1402f and portion 1408 may include any suitable feature(s) 1408f, where such features may interact with one another to hold portions 1402 and 1408 together when portions 1402 and 1408 are rotated about hinge 1405 towards one another. Feature 1402f may interact and engage with a first portion of feature 1408 for clamping about a first tube 102* with a passageway of a cross-section (e.g., diameter) DP′ (see, e.g., FIG. 14B), while feature 1402f may interact and engage with a second portion of feature 1408 for clamping about a second tube 102 with a passageway of a cross-section (e.g., diameter) DP (see, e.g., FIG. 14C) that may be smaller than DP′, such that hinged clip 1400A with stopper 1300A may tightly hold different sized tubes. Alternatively, as shown in FIGS. 14D and 14E, the bottom end of stopper 1300A (e.g., bottom end 1379) may be coupled to (e.g., adhered to) or integrated with (e.g., molded (e.g., double shot molded) with) a top end of a locking mechanism or C-shaped clamp 1400D that may include a first portion 1402′ and a second portion 1408′ for either exposing passageway 1375 of stopper 1300A and clip 1400D for enabling tube 102 to be positioned therein or for closing clamp 1400D about tube 102. As shown, portion 1402′ may include any suitable feature(s) 1402f and portion 1408′ may include any suitable feature(s) 1408f, where such features may interact with one another to hold portions 1402′ and 1408′ together when portions 1402′ and 1408′ are squeezed together. While stopper 1300A may be made of any suitable soft material for comfortably interacting with the inside of intimate exterior of a nostril, each one of locking mechanism or clip 1400A and/or locking mechanism or clamp 1400D may be made of a more rigid material for more firmly grabbing or contracting around a tube for holding the tube in a particular position with respect to the stopper.

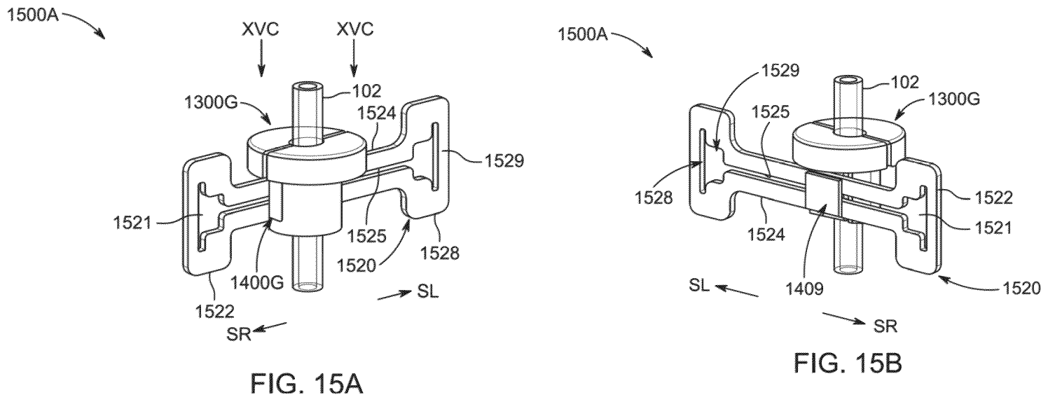

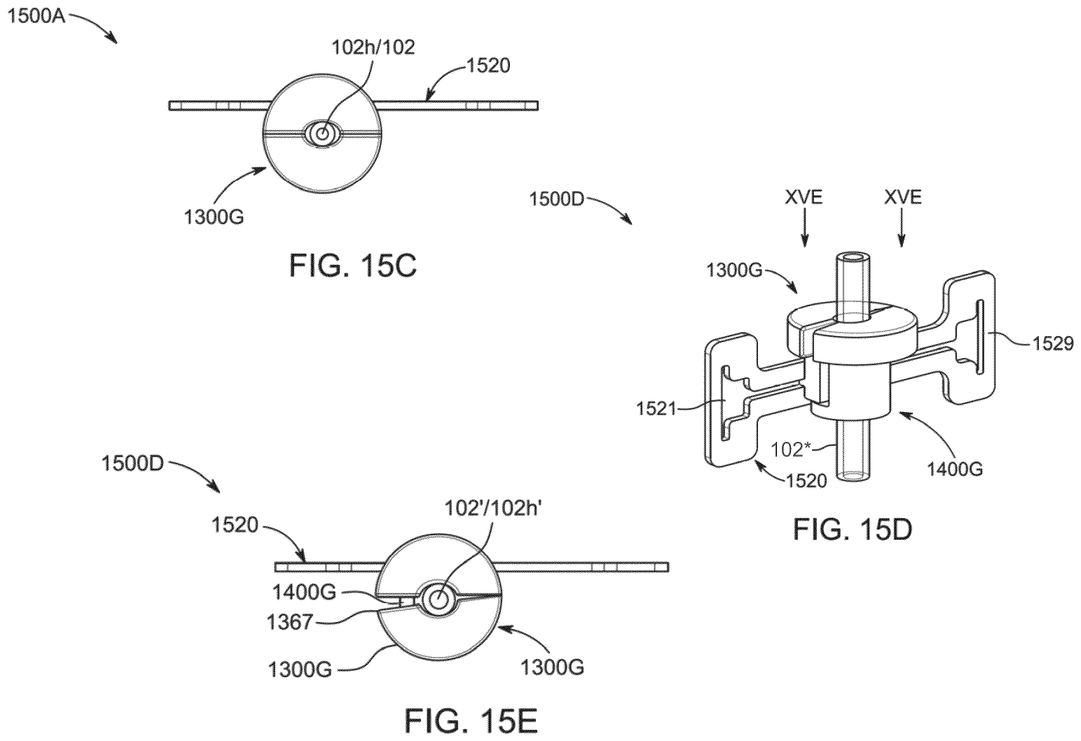

A combined stopper and locking mechanism may be slidably coupled to a mounting subassembly that may be strapped to a patient's head. For example, as shown in FIGS. 15A-15C, 16, 17A, and 17B, a tube holding assembly 1500A may include tube 102 held by a disc shaped stopper 1300G coupled to or integrated with a locking mechanism 1400G, while locking mechanism 1400G (e.g., an extender portion 1409) may be slidably coupled to a mounting subassembly 1520 that may include a mount body 1524 extending between a right end 1522 including an opening 1521 and a left end 1528 including an opening 1529 and a track space 1525 extending between openings 1521 and 1529. An end 1511 of a lower strap 1512 of a strap subassembly 1510 may be passed through opening 1521 and coupled to strap subassembly 1510 with any suitable fastening mechanism 1511f, an end 1513 of an upper strap 1514 of strap subassembly 1510 may be passed through opening 1521 and coupled to strap subassembly 1510 with any suitable fastening mechanism 1513f, another end of lower strap 1512 of strap subassembly 1510 may be passed through opening 1529 and coupled to strap subassembly 1510 with any suitable fastening mechanism, and another end of upper strap 1514 of strap subassembly 1510 may be passed through opening 1529 and coupled to strap subassembly 1510 with any suitable fastening mechanism. A base 1510b of strap subassembly 1510 may extend between a portion of upper strap 1514 and a portion of lower strap 1512 for providing support to a rear portion of patient's head PH when strap subassembly 1510 and mounting subassembly 1520 are coupled to each other and strapped to patient's head PH. A protective flap 1580 may be provided underneath at least a portion of strap subassembly 1510 such that a top portion 1582 of flap 1580 may be folded down over end 1513 and fastening mechanism 1513f (e.g., from the configuration of FIG. 17A to the configuration of FIG. 17B) to prevent the patient from being able to access fastening mechanism 1513f for removing assembly 1500A from patient's head PH and/or such that a bottom portion 1588 of flap 1580 may be folded up over end 1511 and fastening mechanism 1511f (e.g., from the configuration of FIG. 17A to the configuration of FIG. 17B) to prevent the patient from being able to access fastening mechanism 1511f for removing assembly 1500A from patient's head PH. Additionally or alternatively, another flap or the same flap may be provided for covering other fastening mechanisms (e.g., on the other side of the head). Extender portion 1409 of locking mechanism 1400G may be sized and/or shaped to pass through one of openings 1521 and 1529 for accessing track space 1525 along which extender portion 1409 may slide locking mechanism 1400G and thus tube 102 with respect to mounting subassembly 1520 (e.g., in the direction of arrow SL or arrow SR). Disc shaped stopper 1300G may include an opening 1367 that may enable larger tubes (e.g., tube 102*) to be positioned within the passageway of the stopper and locking mechanism (see, e.g., assembly 1500D of FIGS. 15D and 15E). One or more loops 1599 (e.g., hook and fastener equipped ties) may be provided to loop around a strap of subassembly 1510 and a portion of tube 102 for holding tube 102 against strap subassembly 1510 rather than enabling tube 102 to be loose and potentially snag on an ambient element or be grabbed easily by the patient.

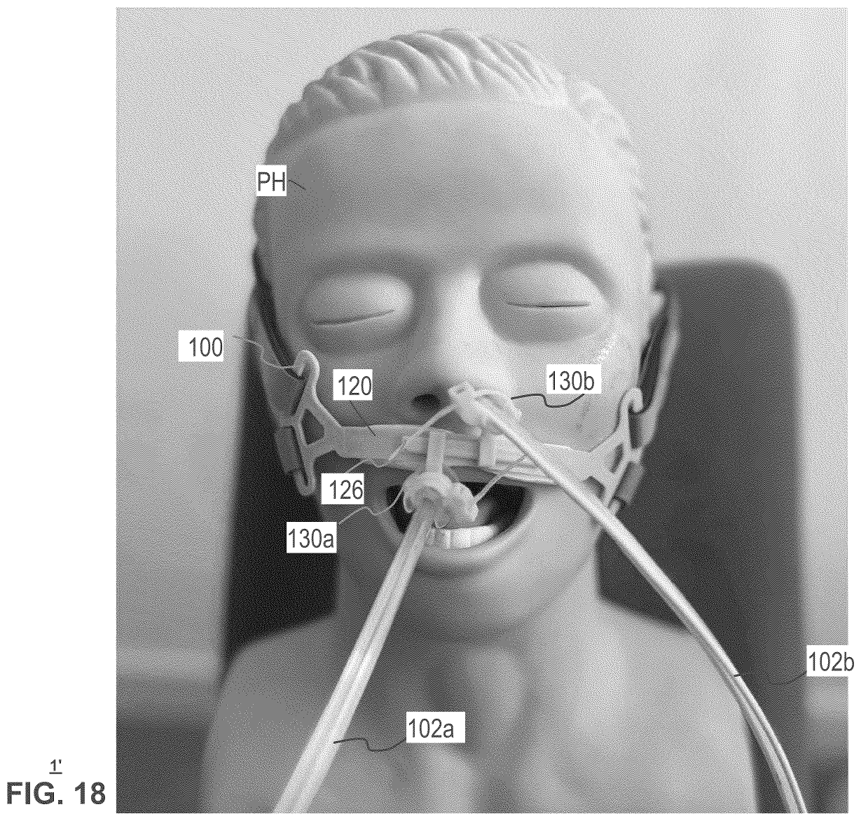

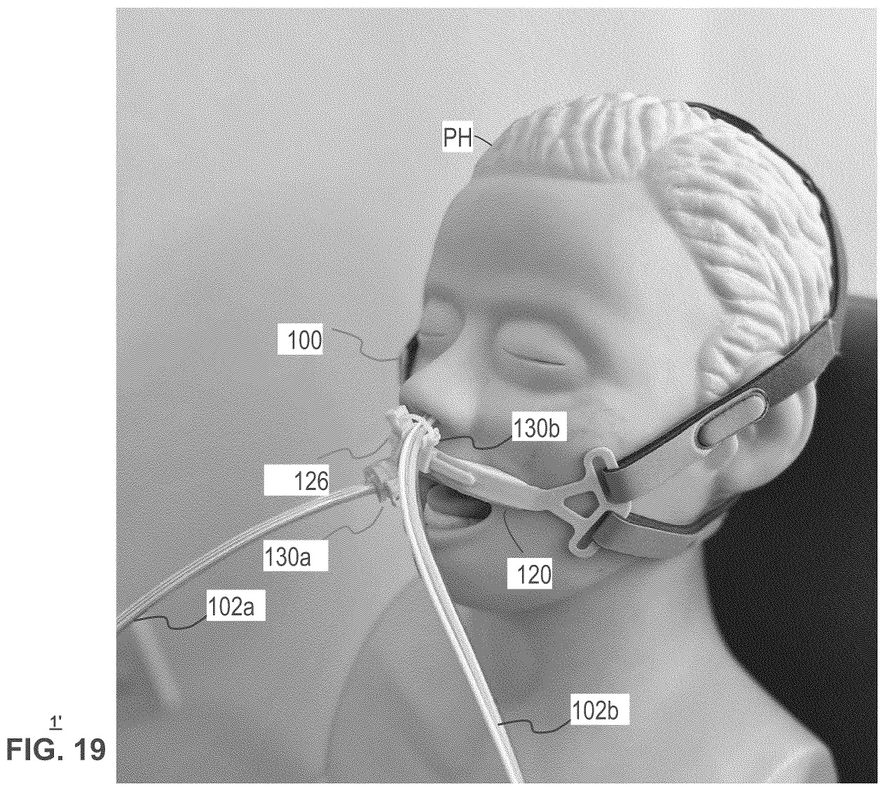

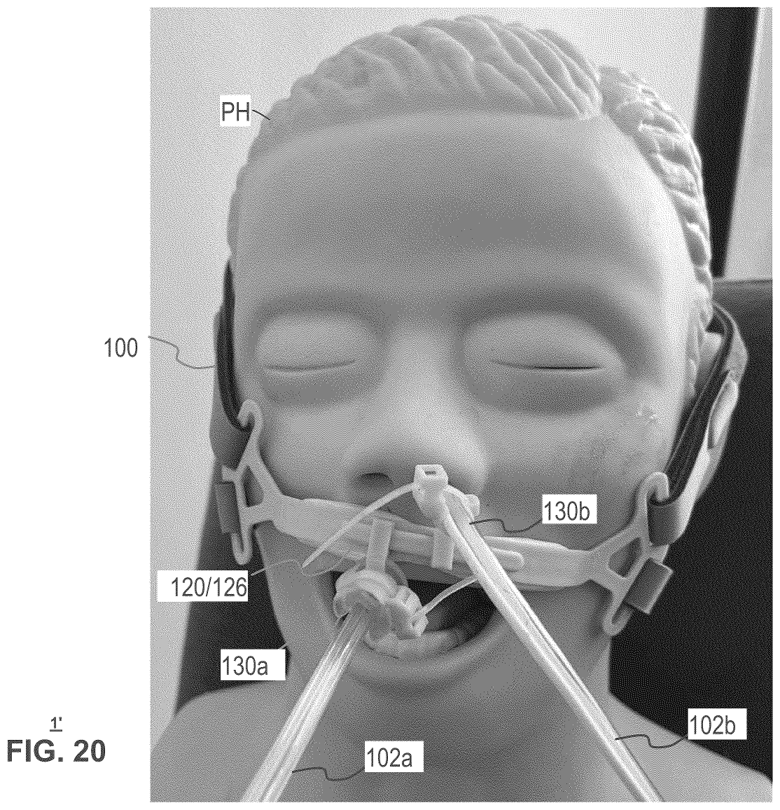

FIGS. 18-20 are different perspective views of a system 1′ that may include patient PH wearing any suitable tube holding assembly (e.g., assembly 100 of FIGS. 1-6) with any suitable mount subassembly (e.g., assembly 120 of FIGS. 1-7) and multiple suitable slider lock subassemblies 130a and 130b, one or each of which may be the same or substantially the same as any suitable slider lock subassembly 130 of FIGS. 8-11 and may or may not include any suitable tube liner (e.g., tube liner 170). As shown, a portion of a first tube 102a may be held within or by or otherwise coupled to slider lock subassembly 130a, while a portion of a second tube 102b may be held within or by or otherwise coupled to slider lock subassembly 130b. Slider lock subassembly 130a may include any suitable slider clamp (e.g., slider clamp 150) that may movably couple slider lock subassembly 130a to mount subassembly 120 of tube holding assembly 100 (e.g., such that slider lock subassembly 130a may be free to slide at least slightly in the direction of one or both of to the left and right with respect to slider clamp mount track body 126 of mount subassembly 120 when the slider clamp of slider lock subassembly 130a is mated with slider clamp mount track body 126). Additionally, as shown, slider lock subassembly 130b may include any suitable slider clamp (e.g., slider clamp 150) that may movably couple slider lock subassembly 130b to mount subassembly 120 of tube holding assembly 100 (e.g., such that slider lock subassembly 130b may be free to slide at least slightly in the direction of one or both of to the left and right with respect to slider clamp mount track body 126 of mount subassembly 120 when the slider clamp of slider lock subassembly 130b is mated with slider clamp mount track body 126).

In some embodiments, slider lock subassembly 130a and slider lock subassembly 130b may be identical or substantially the same but each may be oriented differently with respect to slider clamp mount track body 126 of mount subassembly 120 when the slider clamp of the slider lock subassembly is mated with slider clamp mount track body 126. For example, as shown, slider lock subassembly 130b may be oriented similar to the orientation of FIG. 10 when its slider clamp (e.g., slider clamp 150) is mated with slider clamp mount track body 126, such that a portion of tube 102b may be passed through slider lock subassembly 130b and into a nostril of patient PH (e.g., when the slider clamp of slider lock subassembly 130b is coupled to track body 126 at a position below the nostril), while slider lock subassembly 130a may be oriented 180° to the orientation of FIG. 10 when its slider clamp (e.g., slider clamp 150) is mated with slider clamp mount track body 126, such that a portion of tube 102a may be passed through slider lock subassembly 130a and into the mouth of patient PH (e.g., when the slider clamp of slider lock subassembly 130a is coupled to track body 126 at a position above the mouth). Therefore, the tube clamp (e.g., tube clamp 140) of slider lock subassembly 130a can slide (e.g., with the slider clamp (e.g., slider clamp 150) of slider lock subassembly 130a) at least partially below slider clamp mount track body 126 (e.g., from the perspective of patient PH) to capture an oral tube (e.g., tube 102a) (e.g., hinge 148h and/or the axis of rotation of slider clamp 150 with respect to tube clamp 140 of slider lock subassembly 130a may be positioned at least partially below track body 126), while the tube clamp (e.g., tube clamp 140) of slider lock subassembly 130b can slide (e.g., with the slider clamp (e.g., slider clamp 150) of slider lock subassembly 130b) at least partially above slider clamp mount track body 126 (e.g., from the perspective of patient PH) to capture a nasal tube (e.g., tube 102b) (e.g., hinge 148h and/or the axis of rotation of slider clamp 150 with respect to tube clamp 140 of slider lock subassembly 130b may be positioned at least partially above track body 126). Although shown to be supporting tube 102b entering the left nostril of patient PH, slider lock subassembly 130b may be positioned along track body 126 to support tube 102b such that it may enter the right nostril of patient PH, no matter whether slider lock subassembly 130a remains to the right of slider lock subassembly 130b (e.g., as shown in FIGS. 18-20 (e.g., from the perspective of patient PH)) or whether slider lock subassembly 130b is positioned to the right of slider lock subassembly 130a (e.g., from the perspective of patient PH). Any suitable feature(s) may be provided on mount subassembly 120 and/or on either one or both of slider lock subassemblies 130a and 130b to prevent a slider lock subassembly from sliding off an end of slider clamp mount track body 126 (e.g., all the way to the left and beyond slider clamp mount track left end 1261 for disengaging with track body 126 and/or all the way to the right and beyond slider clamp mount track right end 126r for disengaging with track body 126 (e.g., from the perspective of patient PH)). Additionally or alternatively, any suitable feature(s) may be provided on mount subassembly 120 and/or on either one or both of slider lock subassemblies 130a and 130b (e.g., by slider clamp 150 and/or by track body 126) to prevent a slider lock subassembly from easily popping off of and away from slider clamp mount track body 126 (e.g., out away from the face of the patient) while still enabling an operator (e.g., doctor) to decouple the slider lock subassembly from mount subassembly 120 and recouple the slider lock assembly to mount subassembly 120 without sliding the slider lock assembly off of an end of track body 126 (e.g., to move slider lock subassembly 130a from a first position along track body 126 between slider clamp mount track right end 126r and slider lock subassembly 130b (e.g., as shown in FIGS. 18-20) to a second position along track body 126 between slider clamp mount track left end 1261 and slider lock subassembly 130b (e.g., without sliding slider lock subassembly 130a off of slider clamp mount track right end 126r)). Therefore, a slider lock assembly 130 of system 1′ may capture a tube orally or nasally simply by a mere reversal of the orientation of assembly 130 with respect to track body 126 of tube holding assembly 100 of system 1′. It is to be understood that while FIGS. 18-20 may illustrate subassemblies 130a and 130b oriented differently on track body 126 for enabling tube 102a to enter the mouth while enabling tube 102b to enter a nostril, both subassemblies 130a and 130b may be oriented similarly on track body 126 for enabling tubes 102a and 102b to both enter the mouth or to both enter the same or different nostrils.

In some embodiments, disclosed tube holding devices (e.g., for any suitable tube (e.g., a gastric (e.g., nasogastric or orogastric) or tracheal (e.g., endotracheal (“ET”) (e.g., nasotracheal or orotracheal)) tube)) may have a bite block integrated into a tube holder portion of the device. In one example, the disclosed devices have a tube holder that is side-to-side adjustable along with an ET tube secured by the device. In one example, the disclosed devices have a bite block that is integrally molded as a part of the tube holder. In one example, the disclosed devices have a bite block with one or more features to assist in inserting an ET tube, retaining the ET tube in position once inserted, and accommodating an accessory line that passes within the bite block but outside of the ET tube. These and other objects, features, and advantages of the present disclosure will become apparent to those having ordinary skill in the art upon reading this disclosure.

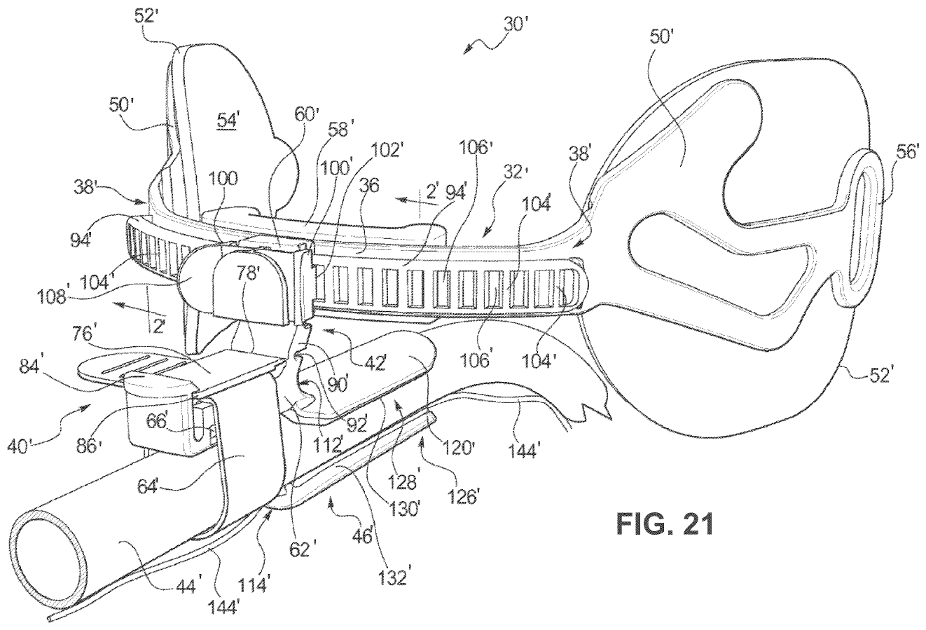

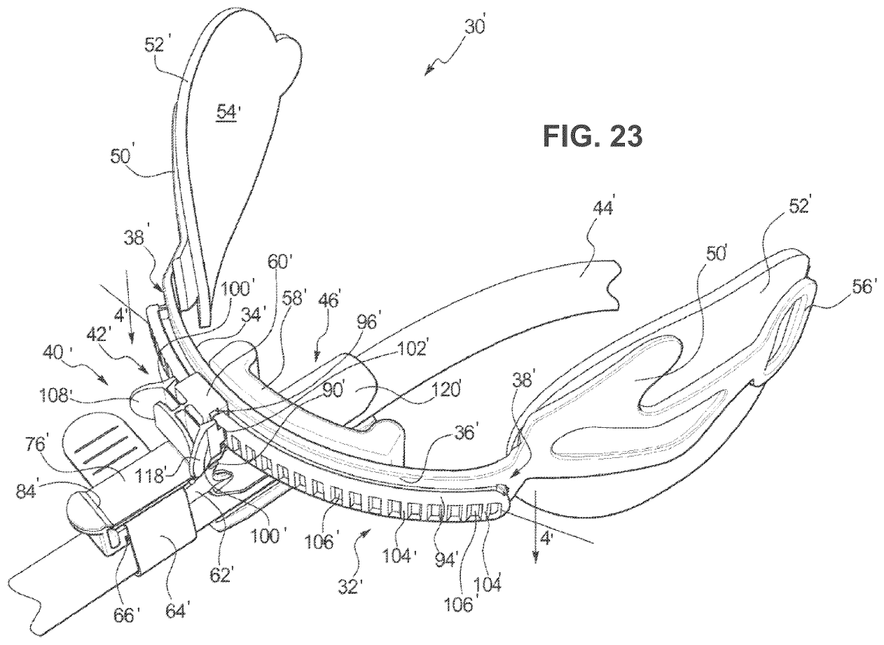

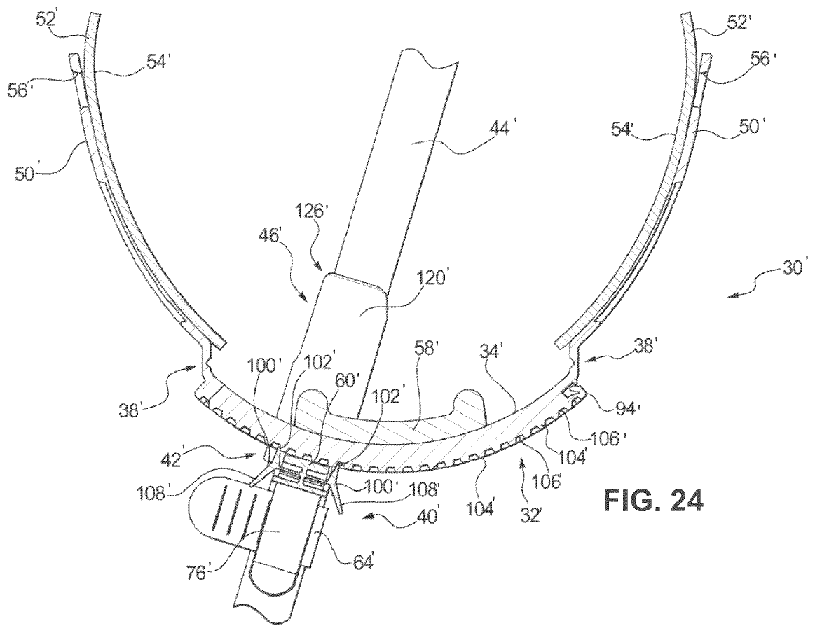

Turning now to the drawings, FIGS. 21-24 show one example of a device 30′ for holding and securing a tube, such as an ET tube, on a patient. Many aspects of the device are disclosed in detail in U.S. Pat. No. 5,490,504. The entire content of the '504 patent is hereby incorporated by reference herein. As shown in FIG. 21, the device 30′ is an ET tube attachment device for securing an ET tube to a patient requiring critical medical care. The disclosed device 30′ generally has a track 32′ that is configured to fit adjacent a lip on a patient's face. In this particular example, the track 32′ is configured to rest above the patient's upper lip. However, the track 32′ can also be configured to rest below a patient's lower lip in another example. In each example, however, the track 32′ is intended to extend laterally or lie horizontally across a portion of the user's face. The track 32′ has a face contacting side 34′ and an exposed side 36′ opposite the face contacting side. The track 32′ also has a pair of opposite ends 38′.

The device 30′ also generally has a tube holder 40′ that is coupled to and slidable along the track 32′ between the opposite ends 38′ on the track. The device 30′ also has a positioning mechanism 42′ that is releasably lockable to allow selective lateral repositioning of the tube holder 40′, as well as a tube 44′ (e.g., endotracheal or other suitable tube) that is held or secured thereby, along the track 32′. The positioning mechanism 42′ is also configured to retain the tube holder 40′ at a selected position along the track 32′. The device 30′ also incorporates a bite block 46′ that is carried by the tube holder 40′ and slidable therewith along the track 32′. Details of the bite block 46′ are described in greater detail below. The bite block 46′ in this example is positioned and spaced vertically below the track 32′ so that the bite block extends between the teeth within a patient's mouth during use. If the track 32′ were below a patient's lower lip, the bite block 46′ would be spaced vertically above the track.

The track 32′ is preferably made of a tough, durable, semi-rigid but somewhat flexible plastic material, such as polyethylene, and is preformed or molded with a curved or arcuate shape to fit on a region adjacent to and along one lip of the patient. Although track 32′ can be positioned along either of the patient's lips, it may be preferable to position the track along the patient's upper lip to avoid having movement of the patient's jaw affect positioning and performance of the device 30′, and particularly the ET tube 44′.

The device 30′ in this example also has a cheek plate 50′ is connected to each of the opposite ends 38′ of the track 32′. A skin friendly cheek pad 52′ is also coupled to each of the cheek plates 50′ on the inside face of each plate. An adhesive layer (not show) can be provided on the face contacting side 54′ of each of the cheek pads 52′. The adhesive layer can also be skin friendly and can help adhere the cheek plates 50′ and track 32′ to the patient's face during use. The cheek plates 50′ and pads 52′ can be curved or contoured to closely follow the curved contour of a patient's face. Each cheek plate 50′ in this example can have one or more strap loops 56′ at or near their free ends. An adjustable head strap 58′ can be coupled to the device 30′ via the strap loops 56′ for securing the device to a patient's head and retaining the track 32′, cheek plates 50′, and cheek pads 56′ on the patient's face. A separate lip pad 58′ can be provided on the face contacting side 34′ of the track 32′ as well. The lip pad can be adhered to the track and can also have a skin friendly adhesive on the rear exposed side to help retain the track in position against the patient's face during use.

As will be evident to those having ordinary skill in the art, the track, cheek plates, cheek pads, and head strap can vary in configuration and construction and yet fall within the scope of the disclosure and claims. The track and cheek plates can be molded as one integrated plastic structure, if desired. The head strap can be formed having any suitable adjustable fastening mechanism, such as a hook and loop structure on a fabric strap. The cheek plates can be formed having any number of configurations and constructions and can utilize a minimum amount of base material (i.e., plastic) and yet function as intended.

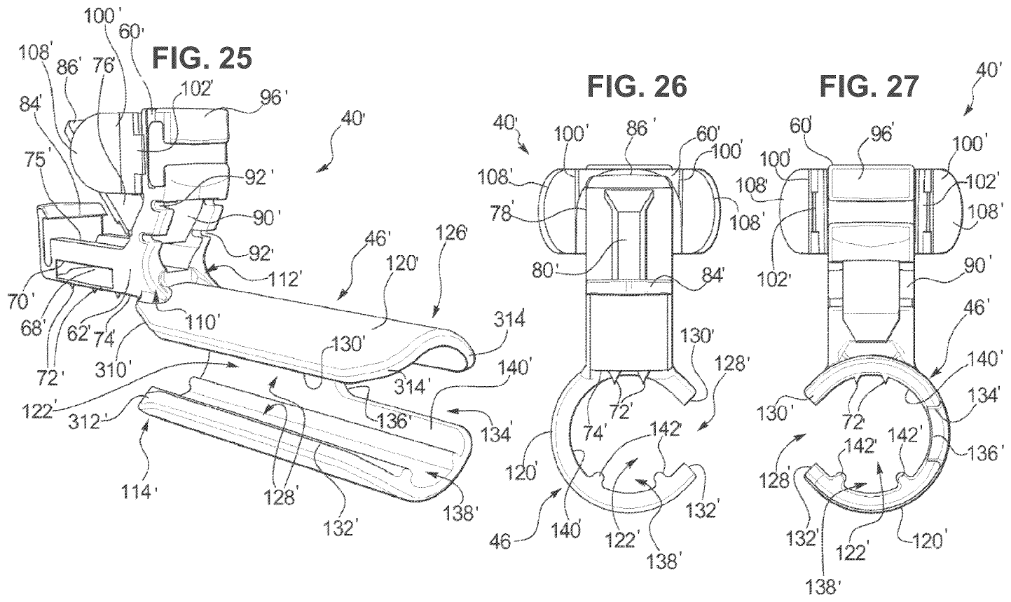

With reference to FIGS. 25-31, the tube holder 40′ generally has a shuttle 60′ that is slidably mounted on the track 32′. The tube holder 42′ also has an arm 62′ that extends in a direction perpendicular to the track 32′ and in a direction away from the exposed or outer surface 36′ of the track. Securement means are provided on the arm 62′ for securing an ET tube 44′ thereto in a direction parallel to the arm. In one example, the securement means can employ a soft, flexible, elongate tube strap 64′ of an elastomeric material. One end of the tube strap 64′ has an enlarged retaining portion 66′. As shown in FIGS. 25′, 28′, and 30′, a slot 68′ for receiving the tube strap 64′ is formed across the arm 62′. The slot 68′ has chamfered or recessed entries 70′ on either side so that the strap 64′ can be threaded through the slot in either direction and the retaining portion 66′ can seat in one of the chamfered entries 70′, fixing that end of the strap to the arm 62′. A free length of the tube strap 64′ extends in a direction transverse to arm 62′ and can be wrapped around the ET tube 44′ as shown in FIG. 21. An adhesive pad 32′ (not shown) or layer, such as a suitable pressure-sensitive adhesive, can be provided on an inner surface of the tube strap 62′ to further restrain the ET tube 44′ from rotational or longitudinal movement when secured against the bottom of the arm 62′. A plurality of sharp, nub-like tangs or spikes 72′ can also be provided on the bottom surface 74′ of the arm 62′. The spikes 72′ can impinge on the exterior surface of the ET tube 44′ to frictionally and/or physically engaging the ET tube and further restraining the tube from movement.

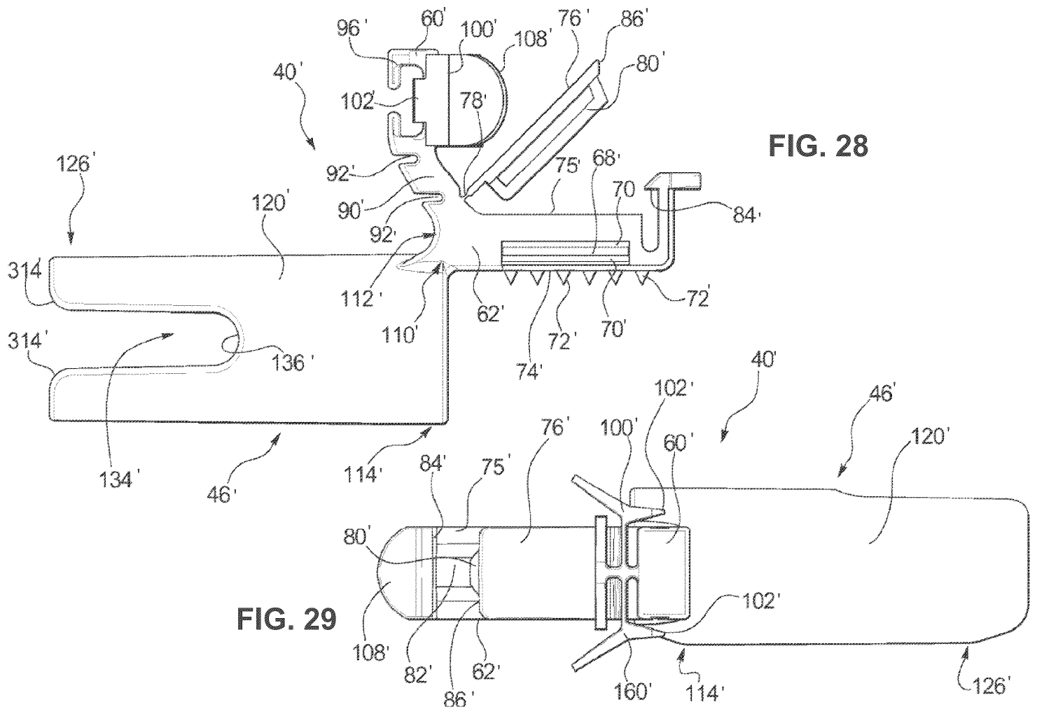

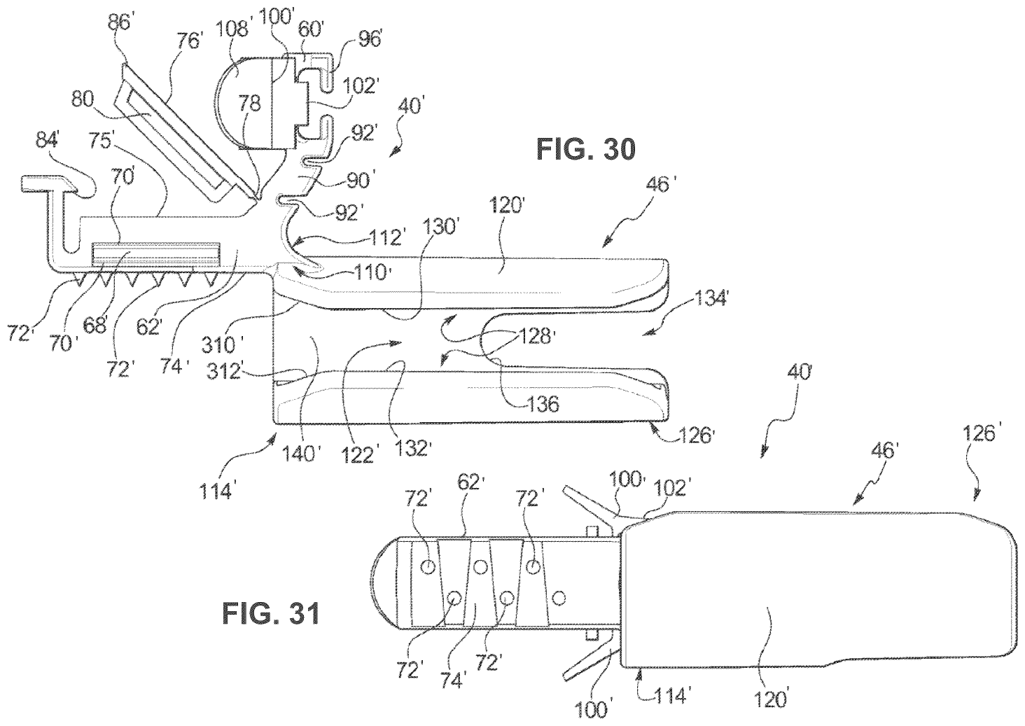

A clamping means is provided on a top side 75′ of the arm 62′, opposite the bottom side 74′. The clamping means in the disclosed example is substantially similar to that disclosed in the aforementioned ‘504’ patent. The clamping means is configured to securely engage and clamp a segment of the tube strap 64′ along its free length, also shown in FIG. 21′. The clamping means can maintain the tube strap 64′ in tension to further help restrain the ET tube 44′ from unintended movement. In the disclosed example, the clamping means is an elongate, cantilevered clamping lever 76′ that is attached via a living hinge 78′ to the tube holder 40′, as shown in FIGS. 28 and 30. The clamping lever 76′ can be pivoted and raised about the living hinge 78′. The free length of the tube strap 62′ can be folded over the top side 75′ of the arm 6′. The clamping lever 76′ can be lowered and locked in place against the arm 62′ to pinch and maintain the tube strap 62′ in tension about the ET tube 44′. A lengthwise or axial rib 80′ is provided in this example and protrudes down from the clamping lever 76′. The top side 75′ of the arm 62′ has a corresponding longitudinal or lengthwise channel or recess 82′ sized to receive the rib 80′ when the clamping lever is closed. The rib 80′ and channel 82′ are provided in order to crimp the tube strap 62′ and help to ensure that the tube strap does not slip from between the arm 62′ and the clamping lever 76′.

In this example, a latching means is also provided to lock and hold the clamping lever 76′ in the locked or clamped condition of FIG. 21. The latching means in one example can have a flexible, resilient catch 84′ provided on the top side 75′ and near the front end of the arm 62′. When the clamping lever 76′ is lowered onto the top side 75′ of the arm 62′, the catch 84′ will flex forward to permit the front edge 86′ of the clamping lever 76′ to snap past the catch 84′. The catch 84′ will then hold and retain the clamping lever 76′ in the closed or clamped position of FIG. 21 to tightly engage the tube strap 62′. To release the ET tube 44′, a person can pull the catch forward away from the clamping lever 76′ until the front edge 86′ can clear the catch 84′. The clamping lever 76′ can then be raised to release the tube strap 62′. This type of latching means allows one to release the ET tube 44′, readjust its position, and then re-secure the tube again without having to replace any components, tape, and the like.

The arm 62′ is connected to the shuttle 60′ by a flexible leg 90′. The flexible leg 90′ in this example has one or more relieved sections 92′ that allow the leg to bend and flex so that the arm 62′ can move slightly relative to the shuttle. Such flexibility can impart a degree of give or yield between the shuttle and the arm so that the track can stay in position on the patient's face even while the patient's involuntary movements may cause movement of the ET tube 44′ and the arm 62′ during use.

As will be evident to those having ordinary skill in the art upon reading this disclosure, the disclosed device 30′ is not to be limited to the particular tube holder construction disclosed herein. The arm, latching means, clamping means, flexible leg, and shuttle can vary in configuration and construction and yet function as intended.

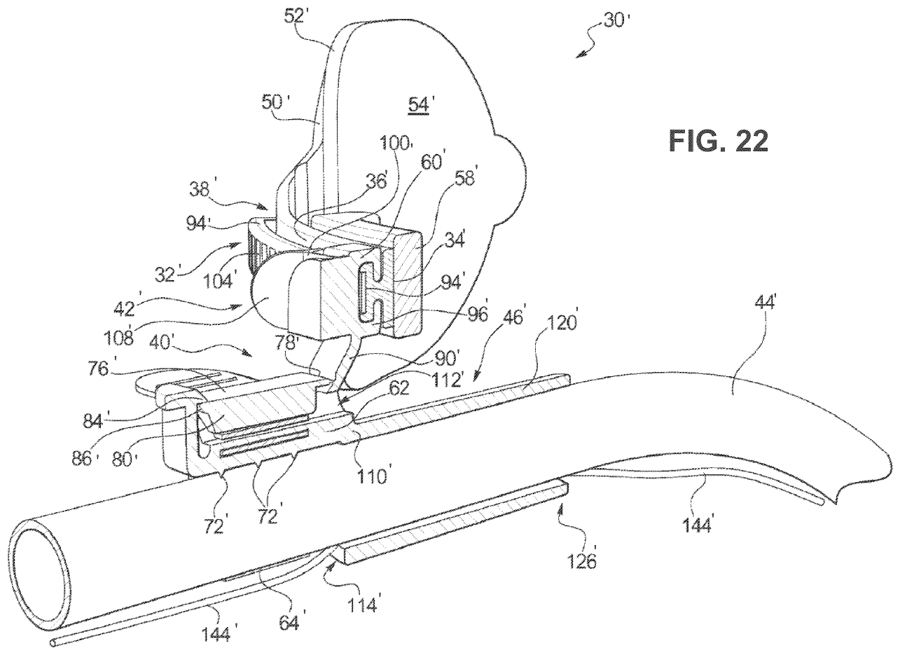

The positioning mechanism 42′ in the disclosed example is substantially similar to that disclosed in the aforementioned ‘504’ patent. In general, the positioning mechanism has two components with one being carried on the track 32′ and the other being carried on the shuttle 60′ of the tube holder 40′. The positioning mechanism 42′ in this example allows selective lateral positioning of the tube holder 40′ and the ET tube 44′ along the track 32′ without having to remove the device 30′ from the patient or the ET tube from the device. In this example, one component of the positioning mechanism is a rail 94′ disposed on the exposed side 36′ of the track 32′. The rail 94′ is generally T-shaped in cross-section (i.e., when viewed from the side of the device 30) as shown in FIG. 22′. The rail 94′ is complementary shaped relative to a back side of the shuttle 60′. In this example, the shuttle has a C-shaped sliding retainer 96′ that slides along, captures, and engages the rail. The T-shaped rail 94′ and C-shaped retainer 96′ can fit snuggly to one another but without impeding lateral sliding movement of the shuttle 60′ along the track 32′. One or more stops (not shown) may be provided at each of the opposite ends 38′ of the track 32′ to keep the shuttle 60′ from sliding off either end of the rail 94′ during use. These stops can allow the shuttle to be snapped onto one end of the track 32′ during assembly of the device 30′.

The positioning mechanism also includes a locking means, in this example also partly on the shuttle 60′ and partly on the track 32′, to positively lock and hold the tube holder 40′ and ET tube 44′ in the selected position of adjustment on the track. In one example, as shown in FIGS. 21-24, the locking means has a pair of dogs or flex fingers 100′ oriented generally perpendicular to the track 32′. The flex fingers 100′ are resiliently connected to the shuttle 60′ and have prongs 102′ that project rearward toward the track 32′. The prongs 102′ are positioned to engage any one of a plurality of ratchet teeth 104′ provided on a front face of the rail 94′. When the flex fingers 100′ are in an unflexed condition, the prongs 102′ seat in recesses 106′ between the teeth 104′. Grip ends 108′ on the flex fingers 100′ can be squeezed together, which spreads the flex fingers outward away from one another and disengages the prongs 102′ from the ratchet teeth 104′. When disengaged, the tube holder 40′ and shuttle 60′ can slide along the rail 94′ to a desired position along the track 32′. This can be done to allow medical personnel to access the patient's mouth without having to remove the device 30′ or the ET tube 44′. When pressure on the grip ends 108′ the flex fingers 100′ is released, the prongs 102′ will return and engage the ratchet teeth 104′ to retain the tube holder 40′ in the selected position along the track 32′.

As will be evident to those having ordinary skill in the art, the positioning mechanism components, including the locking means, the rail, and the shuttle retainer, can vary from the example shown and described herein. The details of the structure and function of these aspects of the device 30′ can be varied and yet function as intended. The device 30′ has a tube holder 40′ that is side-to-side adjustable along the track, even when the device is attached to a patient. Thus, the ET tube 44′ can be move from one side to another within the patient's mouth to allow medical personnel to clean the patient's teeth or attend to other medical care without having to completely remove the device 30′ from the patient and without having to remove the ET tube 44′ from the device. This makes caring for the patient simpler and easier.

According to the teachings of the present disclosure, the bite block 46′ is integrated into the tube holder, as best illustrated in FIGS. 25-31. In one example, the bite block can be integrally molded from a suitable plastic or similar material as a part of the tube holder 40′. This can render the entire tube holder 40′ a one-piece unit inclusive of the bite block 46′, the arm 62′, the shuttle 60′, the flex fingers 100′, and the clamping mechanism for the tube strap. In one alternate example, the bite block 46′ could be a separate element that is configured to attach to the tube holder. This could be done utilizing a complementary snap connection between bite block and holder or utilizing an adhesive, fasteners, or the like. In such an example, the bite block could be utilized on a patient where the medical personnel determine such usage beneficial. The bite block could also be removed in such an example if the medical personnel determined that the bite block should not be used for some reason.

In the disclosed example, the bite block 46′ is molded to a rear end 110′ of the arm 62′ opposite the catch 84′. A molded joint 112′ connects the bite block 46′ to the arm 62′ in this example. The molded joint 112′ can vary in configuration and construction. The intent is for the connection to be robust and durable so that the bite block 46′ remains attached to the tube holder 40′, as long as intended. The joint 112′ can also be positioned and configured to connect the bite block 46′ to the tube holder 40′ at a number of different locations on both the bite block and holder. In this example, the molded joint 112′ is on the rear end 110′ of the arm 62′ and connects to a forward end 114′ of the bite block 46′. Also in this example, the bite block 46′ is positioned spaced vertically below the track 32′. In other configurations, as noted above, it is possible that the bite block be positioned and spaced above the track. The positioning of the bite block 46′ in this example is such that the ET tube 44′ can still be retained in place by the tube strap 64′ and by the spikes 72′, which are still exposed forward of the bite block on the bottom side 74′ of the arm 62′.

Features of the bite block 46′ are depicted in FIGS. 25′, 26′, and 28′. The bite block 46′ has a generally tubular wall 120′ having a substantially cylindrical shape with a length and a central axis. A central opening 122′ extends along the length of the bite block 46′ and the wall 120′ has a pair of opposite open ends including the aforementioned forward end 114′ and a rear or working end 126′. The working end portion of the wall 120′ of the bite block extends in the direction of the face contacting side 34′ of the track 32′, i.e., in a rearward direction and extends further rearward beyond the surface or face of the face contacting side.

In the disclosed example, the bite block 46′ has an insertion slot 128′ through the wall 120′. The insertion slot 128′ extends along the entire length of the bite block 46′. The wall 120′ can be formed of a suitable plastic material so that the bite block 46′ is sufficiently flexible to allow an ET tube 44′ to be inserted laterally into the central opening 122′ through the insertion slot 128′. The flexibility of the wall 120′ can allow the width of the slot 128′, and thus the diameter of the wall, to expand when inserting the tube and then to spring back to the normal at rest slot width and wall diameter once the tube is fully inserted. The insertion slot 128′ is defined between spaced apart free edges 130′ (upper) and 132′ (lower) of the wall 120′. The free edges 130′, 132′ confront one another along the length of the bite block 46′ and define the width of the slot 128′. The wall 120′ also has a blind slot 134′ that is formed in the working end portion of the bite block 46′. The blind slot 134′ is open at the working end 124′ of the wall 120′ and terminates only part way along the wall at a closed or blind end 136′. Thus, unlike the insertion slot 128′, the blind slot 134′ extends only part way along the length of the wall 120′. The blind slot is also positioned generally opposite the insertion slot 128′ in the wall. The blind slot 134′ can add some resilience and flexibility to the wall 128′. Thus, during use, the wall 120′ can give slightly if a patient were to occasionally exert a great amount of force upon the bite block 46′ during use. This would divert some of the load or absorb some of the energy from such a force through the wall 120′ instead of directly to the patient's jaw and/or teeth.

The bite block 46′ can also include one or more features to accommodate accessory lines passing through the bite block adjacent the ET tube 44′. Such an accessory line channel can extend the length of the bite block and be formed between the wall and the ET tube within the central opening of the bite block. In this example, the bite block 46′ has an accessory line channel 138′ that extends the length of the bite block 46′ within the central opening 122′. The accessory line channel 138′ can be formed, at least in part on an inner surface 140′ of the wall 120′. The accessory line channel 138′ can also be formed in part by the outer surface of the ET tube 44′, depending on the configuration and construction of the channel. In this example, the accessory line channel 138′ is formed between the inner surface 140′ of the wall 120′ and the outer surface of the ET tube 44′. Two spaced apart ribs 142′ extend along the length of the bite block 46′ on the inner surface 140′ of the wall 120′. The channel would be defined between the ribs and between the outer ET tube surface and the inner surface 140′ of the wall 120′. The ribs 142′ create a space between the inner surface 140′ and the ET tube 44′. An accessory line, such as a pilot tube 144′ (see FIG. 21) for inflating a retention cuff or balloon (not shown) on the end of the ET tube, can be run along the outside of the ET tube 44′ and through the bite block 46′ along the channel 138′ without being pinched off, closed, or crushed.

More than one accessory line channel can be formed along the inside of the bite block 46′. Three or more spaced apart ribs can be provided to define two or more separate channels, if desired. Also, two or more accessory lines can be run along the same channel, if desired. The accessory lines can provide any type of additional line function needed to treat a patient, including but certainly not limited to the pilot line 144′. Also, the configuration, construction, and performance features of the bite block can vary from the bite block 46′ described above. For example, the free edges 130′, 132′ of the insertion slot 128′ on the bite block 46′ can be varied in shape and contour so as to help the wall 120′ retain a generally cylindrical shape, even when a patient's teeth exert a substantial crushing force on the bite block during use. The upper free edge 130′ could have a first contoured shape and the lower free edge 132′ could have a corresponding second contoured shape configured to close against and engage the first contoured shape if a sufficient clamping force is exerted on an outer surface of the bite block.

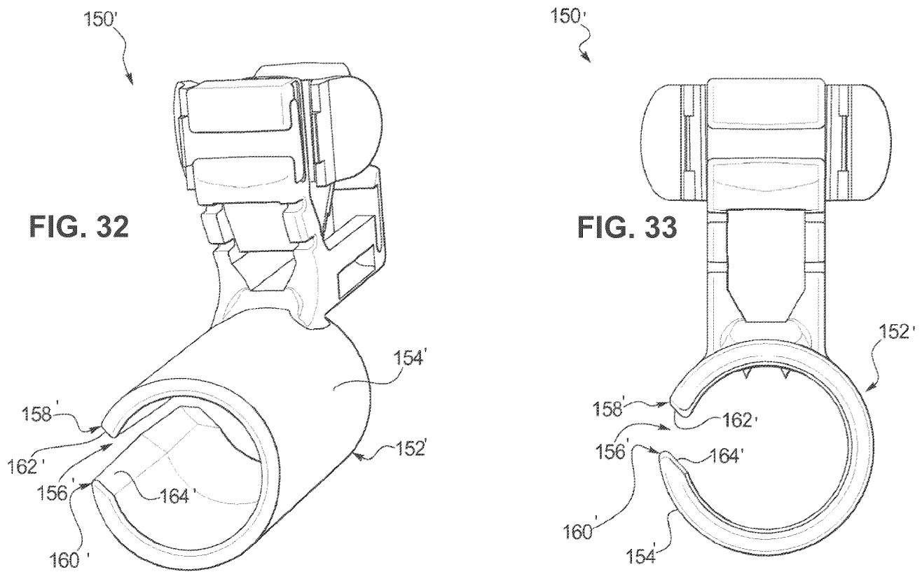

For example, FIGS. 32 and 33 show a tube holder 150′ that is substantially identical to the earlier described tube holder 40′. In this example, the tube holder 150′ has a bite block 152′ that also has a tubular shape with a substantially cylindrical wall 154′. However, the bite block 152′ has a simpler configuration with no accessory line channel and no blind slot. The bite block 152′ does have an insertion slot 156′ along a length of the slot and through the wall 154′. Free edges 158′, 160′ of the insertion slot 156′ are contoured in this example and are closer together resulting in a narrower insertion slot 156′ than the slot 128′ of the bite block 46′. The upper free edge 158′ has an angled or beveled edge forming a flat face 162′ and the lower free edge 160′ has a corresponding angled or beveled edge defining a complementary flat face 164′. If a patient were to apply sufficient bite force on the bite block, the flat faces 162′, 164′ would engage and mate with one another under less of a load than the bite block 46′ because the free edges 158′, 160′ are closer together. Also, the flat faces 162′, 164′ being in contact will help prevent the free edges 158′, 160′ from bypassing one another and thus will help prevent the wall 154′ from collapsing beyond its cylinder shape.

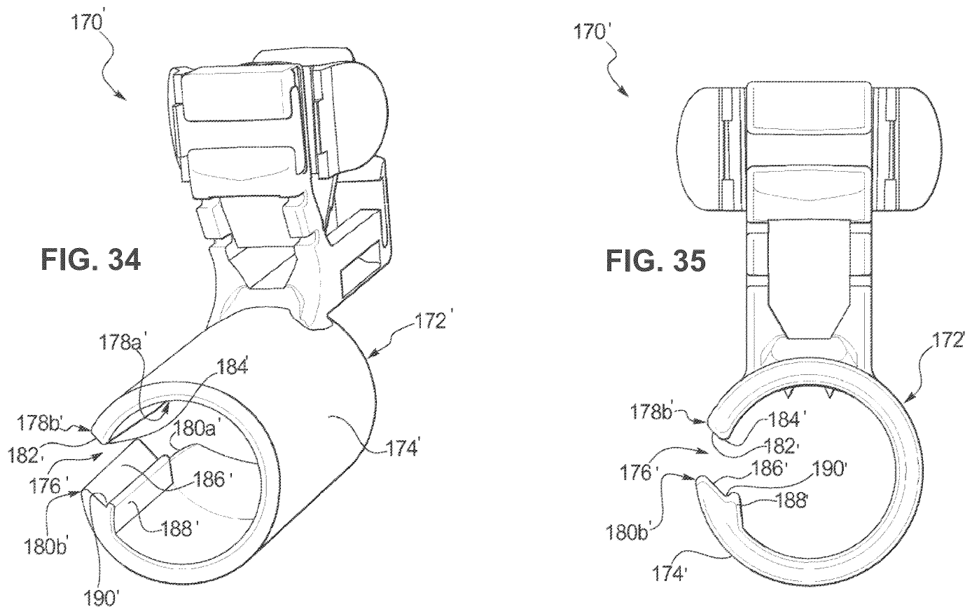

FIGS. 34 and 35 show another example of a tube holder 170′ that is also substantially identical to the earlier described tube holder 40′. In this example, the tube holder 170′ has a bite block 172′ that also has a tubular shape with a substantially cylindrical wall 174′. The bite block 172′ also has a simpler configuration with no accessory line channel and no blind slot in comparison to the bite block 46′. The bite block 172′ does have an insertion slot 176′ along a length of the slot and through the wall 174′. Free edges of the insertion slot 176′ are also contoured in this example. Forward edge portions 178a, 180a are not contoured and are further apart from one another leaving a greater slot width at a forward end of the insertion slot 176′. Rearward edge portions 178b, 180b of the free edges are closer together resulting in a narrower width slot over part of the insertion slot 176′ than the slot 128′ of the bite block 46′.

The upper free edge portion 178b has an angled or beveled edge forming a flat face 182′ and a convex V-shaped terminus 184′ along the free edge portion. The lower free edge portion 180b has a corresponding angled or beveled edge defining a complementary flat face 186′ and also has a stepped ridge 188′ protruding into the central opening 122′. The stepped ridge cooperates with the complementary flat face 186′ to create a concave V-shaped trough or groove 190′ along the free edge portion 180b. If a patient were to apply sufficient bite force on the bite block 170′, the bottom terminus 184′ would engage and seat in the groove 190′ to help prevent the two free edges 178b, 180b from bypassing one another and thus to help prevent the wall 172′ from collapsing beyond its cylinder shape. The contoured free edge portions in this example may be more robust in this regard than those of the bite block 150′.

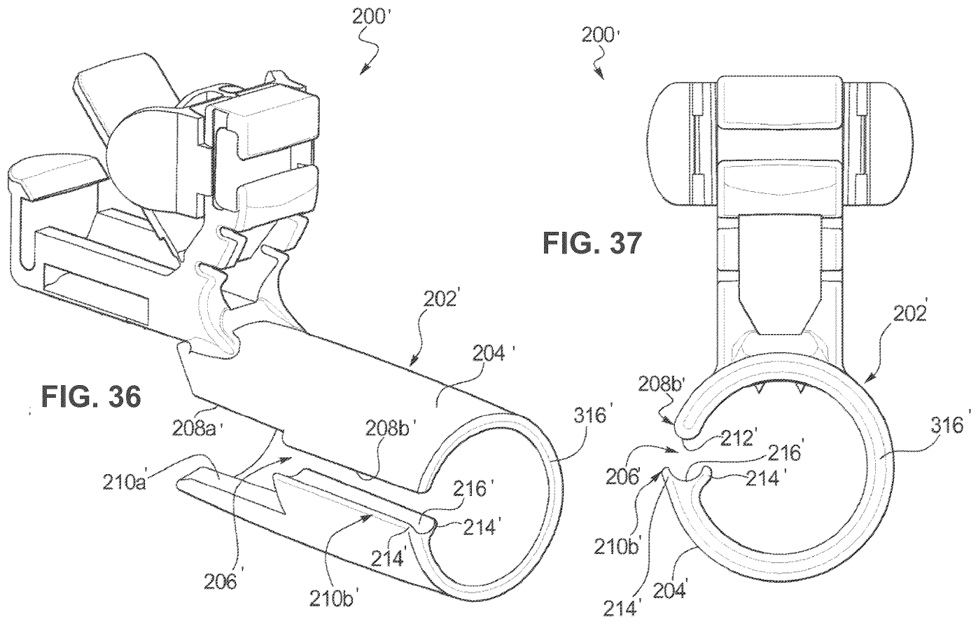

FIGS. 36 and 37 show another example of a tube holder 200′ that is also substantially identical to the earlier described tube holder 40′. In this example, the tube holder 200′ has a bite block 202′ that is substantially the same as the bite block 170′, other than the contoured edge portions. In this example, the bite block 202′ also has a tubular shape with a substantially cylindrical wall 204′. The bite block 202′ also has no accessory line channel and no blind slot in comparison to the bite block 46′. The bite block 202′ does have an insertion slot 206′ along a length of the slot and through the wall 204′. Free edges of the insertion slot 206′ are also contoured in this example. Forward edge portions 208a, 210a are not contoured and are further apart from one another leaving a greater slot width at a forward end of the insertion slot 206′. Rearward edge portions 208b, 210b of the free edges are closer together resulting in a narrower width slot over part of the insertion slot 206′ than the slot 128′ of the bite block 46′.

The upper free edge portion 208b has a rounded convex contour forming a rounded face 212′ along the free edge portion. The lower free edge portion 210b has a flange 214′ that is wider than the wall 204′ thickness. The upward facing side of the flange 214′ has a concave rounded groove sized to complement the rounded face 212′ of the upper edge portion 208b. If a patient were to apply sufficient bite force on the bite block 200′, the rounded face 212′ would engage and seat in the rounded groove 216′ to help prevent the two free edges 208b, 210b from bypassing one another and thus to help prevent the wall 202′ from collapsing beyond its cylinder shape. The contoured free edge portions in this example also may be more robust in this regard than those of the bite block 150′.

The bite block in other examples can have alternative concave and convex complementary shape, or other complementary contoured shapes that can engage and mate with one another. The disclosure is not limited only to these examples described herein.

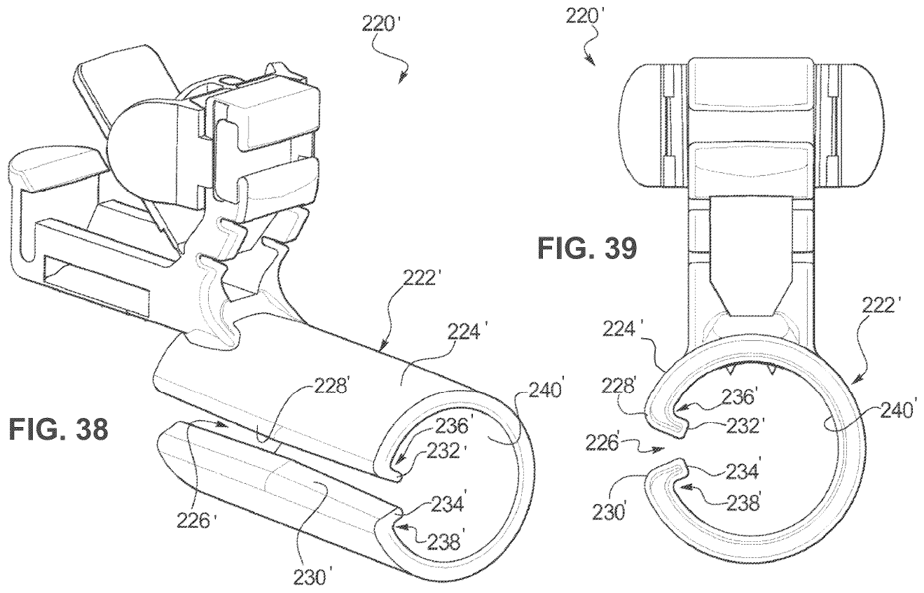

For example, FIGS. 38 and 39 show a tube holder 220′ that is also substantially identical to the earlier described tube holder 40′. In this example, the tube holder 220′ has a bite block 222′ that also has a tubular shape with a substantially cylindrical wall 224′. The bite block 222′ has a simpler configuration with no blind slot in comparison to the tube holder 46′. The bite block 222′ does have an insertion slot 226′ along a length of the slot and through the wall 224′. Free edges 228′, 230′ of the insertion slot 226′ are contoured in this example and are again closer together resulting in a narrower insertion slot 226′ than the slot 128′ of the bite block 46′. Both the upper free edge 228′ and lower free edge 230′ have mirror image flanges 232′, 234′ that extend radially inward into the central opening of the bite block 220′.

The flanges 232′, 234′ make the free edges 228′, 230′ wider than the wall 224′ thickness. The flanges 232′, 234′ are configured to close against and engage one another when a sufficient clamping force is exerted on the bite block, which is less than that of the bite block 46′, again because the free edges 228′, 230′ are closer together. Also, the flanges 232′, 234′ being wider than the wall 224′ thickness will help prevent the free edges 228′, 230′ from bypassing one another and thus will help prevent the wall 224′ from collapsing beyond its cylinder shape. Each of the flanges 232′, 234′ in this example also creates an accessory line channel 236′, 238′ along an inner surface 240′ of the wall 224′ adjacent the flanges within the central opening of the bite block 220′. It is possible that only one of the flanges is configured to form or define such an accessory line channel.

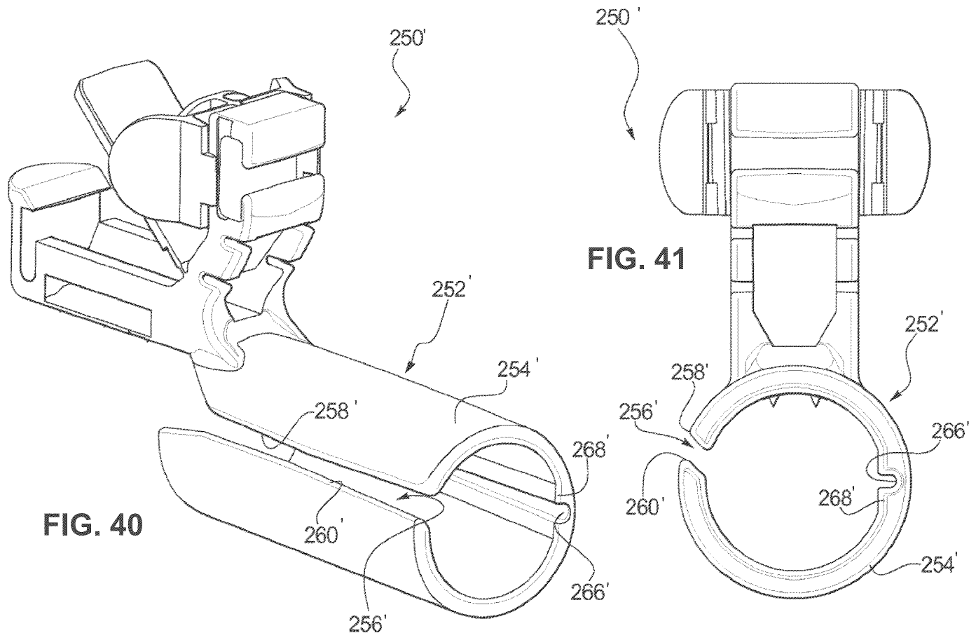

FIGS. 40 and 41 show another alternate example of a tube holder 250′ that is substantially identical to the earlier described tube holder 40′. In this example, the tube holder 250′ has a bite block 252′ that also has a tubular shape with a substantially cylindrical wall 154′. The bite block 252′ also has no accessory line channel and no blind slot. The bite block 252′ does have an insertion slot 256′ along a length of the slot and through the wall 254′. Free edges 258′, 260′ of the insertion slot 256′ are spaced apart and contoured in a manner that is essentially identical to the free edges 158′, 160′ on the bite block 152′ of the tube holder 150′ described previously. The bite block 250′ also has a living hinge 266′ or relief in the wall 254′ generally opposite the insertion slot 256′ and extending a length of the bite block. The living hinge can be formed in a thickened portion 268′ of the wall as shown. Alternatively, the entire wall can be thicker, other than the living hinge 266′. The living hinge can be provided to allow for easier flexing of the wall to insert the ET tube. The thicker wall material can provide a more robust bite block while the living hinge still permits easy ET tube insertion via the insertion slot.

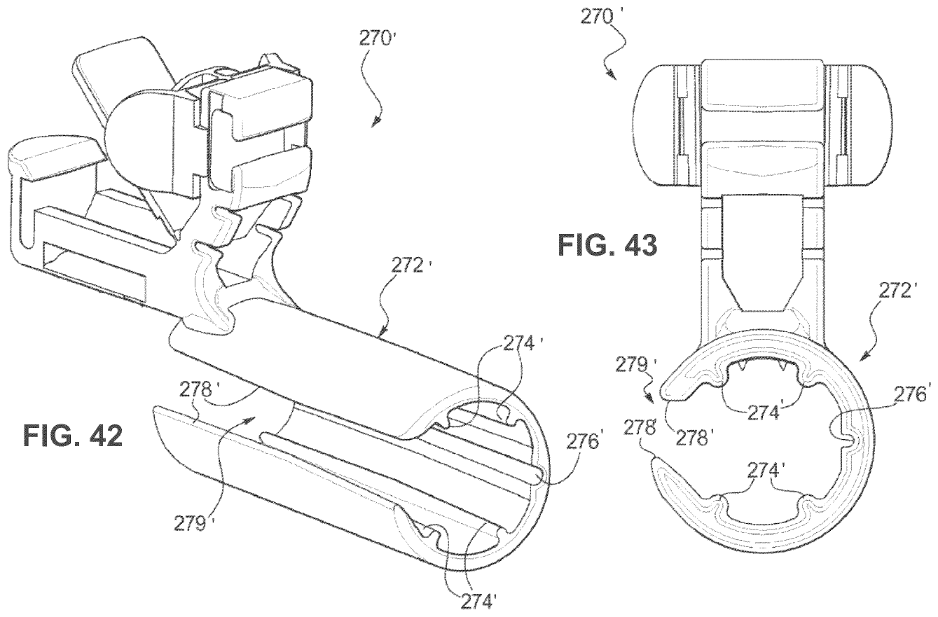

FIGS. 42 and 43 show yet another example of a tube holder 270′ with features similar to earlier described bite block features. In this example, the tube holder 270′ has a bite block 272′ with two sets of ribs 274′ forming two accessory line channels, one above the ET tube and one below the ET tube. The ribs 274′ are otherwise similar to the ribs 142′ described previously. The bite block 272′ also has a living hinge 276′ similar to the living hinge 266′ in the previous example. Also in this example, free edges 278′ of the bite block 272′ along an insertion slot 279′ can be configured to contact one another upon compression of the bite block. In this example, the free edges 278′ can also be configured so that one of the free edges slips past the other until it contacts one of the ribs 274′, which can act as a stop to prevent further compression.

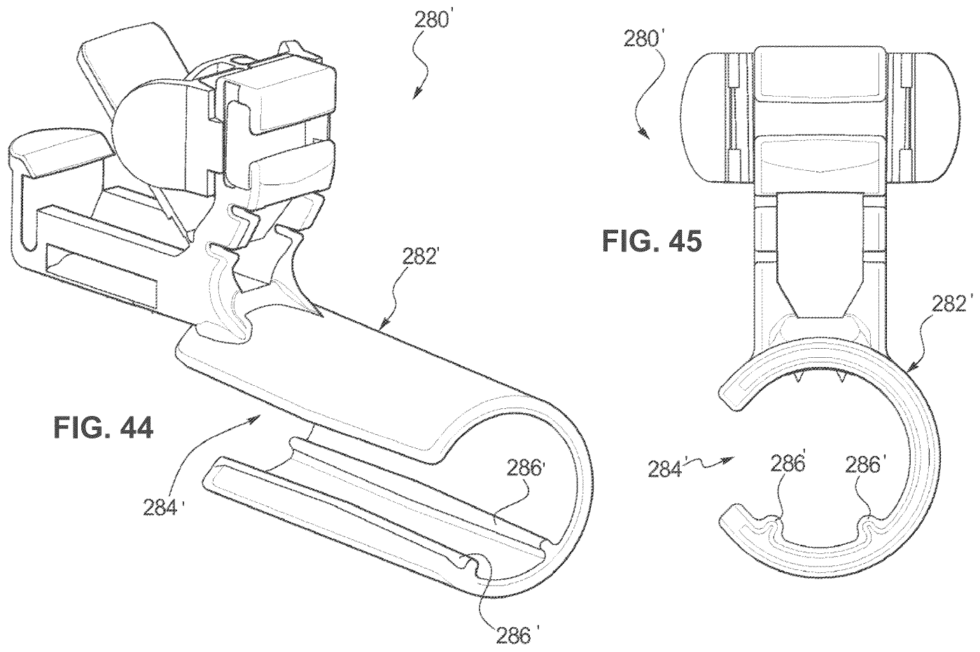

FIGS. 44 and 45 show still another alternate example of a tube holder 280′ with features similar to earlier described bite block features. In this example, the tube holder 280′ has a bite block 282′ with an insertion slot 284′ similar to the slot 128′ of the bite block 46′ with a wide slot width. This bite block 282′ has no blind slot opposite the insertion slot 284′ but does have two spaced apart ribs 286′ forming a single accessory line channel within the bite block.

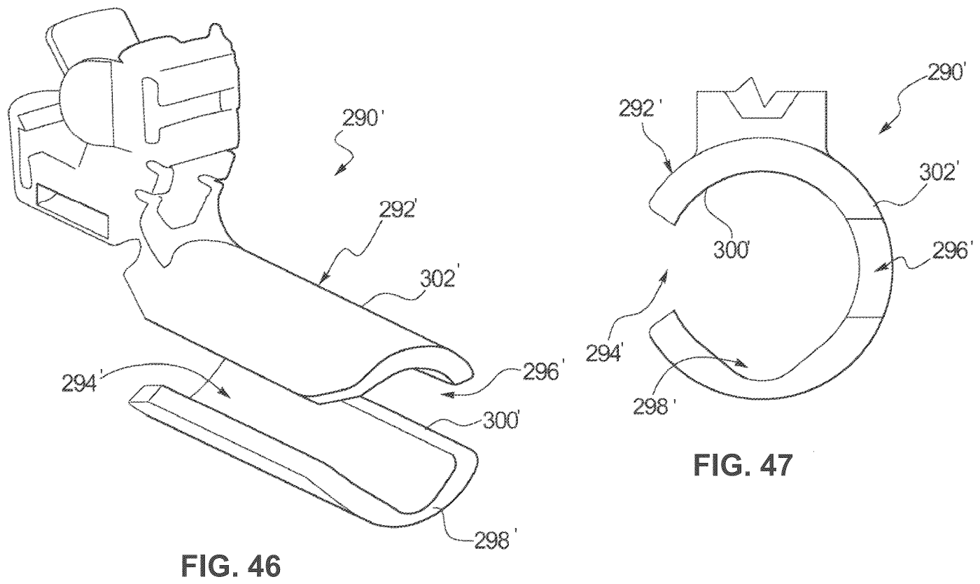

FIGS. 46 and 47 show still another alternate example of a tube holder 290′ with most features similar to earlier described bite block features. In this example, the tube holder 290′ has a bite block 292′ with an insertion slot 294′ similar to the slot 128′ of the bite block 46′ with a wide slot width. This bite block 282′ also has a blind slot 296′ similar to the blind slot 134′ on the bite block 46′ and positioned opposite the insertion slot 294′. The bite block 292′ has a groove 298′ formed along a length of the bite block and recessed into an inner surface 300′ of the bite block wall 302′. The groove 298′ creates an accessory line channel that recessed into the wall 302′, instead of using protrusions to create space between the inner wall and an ET tube in the bite block, as in prior examples. The groove can be V-shaped in cross-section or can have a rounded, semispherical, or other shape as well.

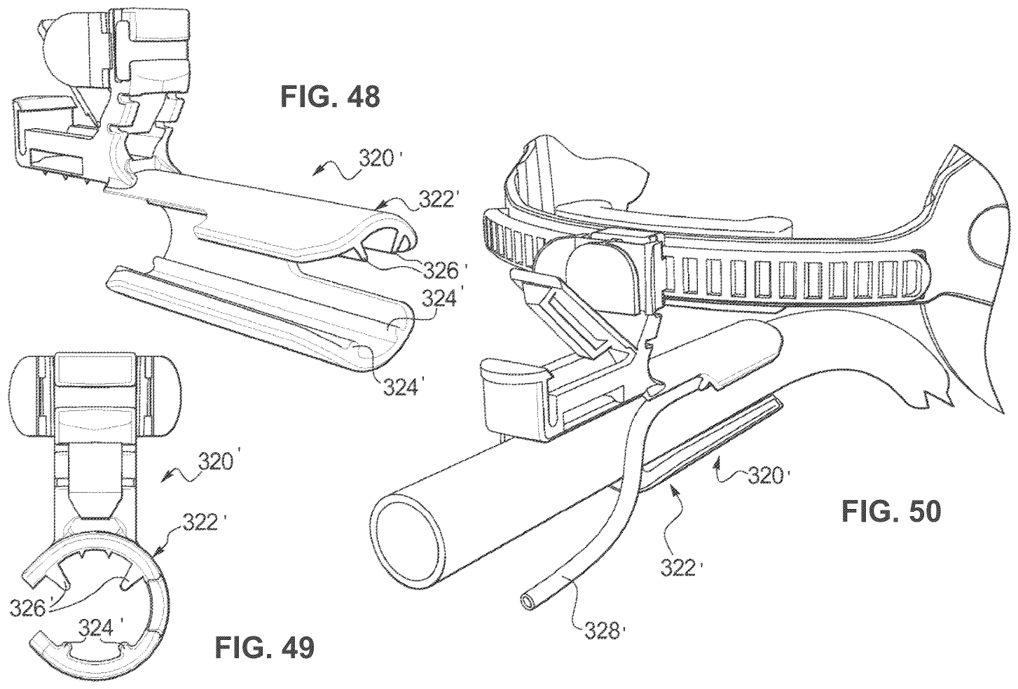

FIGS. 48-50 show yet another example of a tube holder 320′ that is nearly identical to the tube holder 270′ described above. In this example, the tube holder 310′ has a bite block 322′ with two sets of ribs 324′ and 326′ forming two accessory line channels, one above the ET tube and one below the ET tube. In this example, the upper ribs 326′ are longer than the lower ribs 324′. The longer upper ribs provide a deeper accessory line channel between them. In one example, as shown in FIG. 50′, the accessory line can be a subglottic suction line 328′ that communicates with the ET tube. Such a tube may have a larger diameter than a conventional pilot line and require a deeper channel. When the bite block 322′ is compressed during use, the rib length can determine how much crush is imparted on the accessory lien, such as the subglottic suction line.

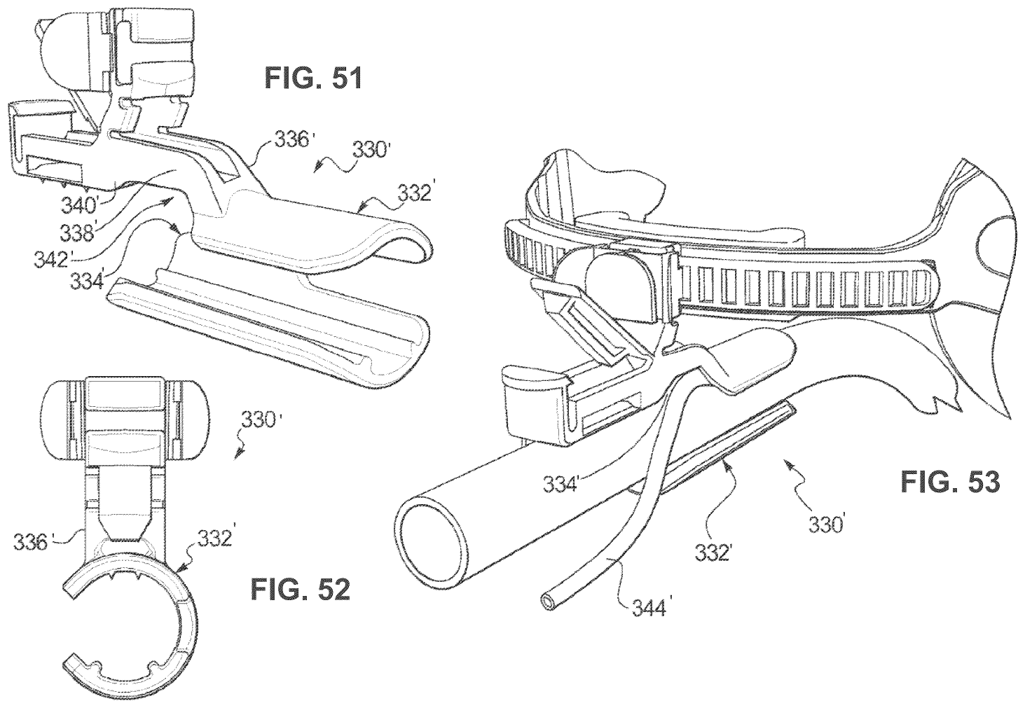

FIGS. 51-53 show still another example of a tube holder 330′ that is substantially the same as many of the prior described example. In this example, the tube holder 330′ has a bite block 332′ that is substantially the same as the bite block 46′. However, the bite block has a second scallop or blind slot 334′ on the front end of the bite block and opposite the insertion slot 336′. The tube holder 330′ also has a revised joint 336′ that extends the bite block 332′ a bit more rearward from the arm 340′ than prior described tube holders. This extended joint 336′ in combination with the blind slot 338′ creates a gap or clearance 342′ under the arm and forward of the bite block 332′. As shown in FIG. 53, a second accessory line 344′, such as a subglottic suction line, can exit the ET tube and pass under the arm 340′ and joint 336′.

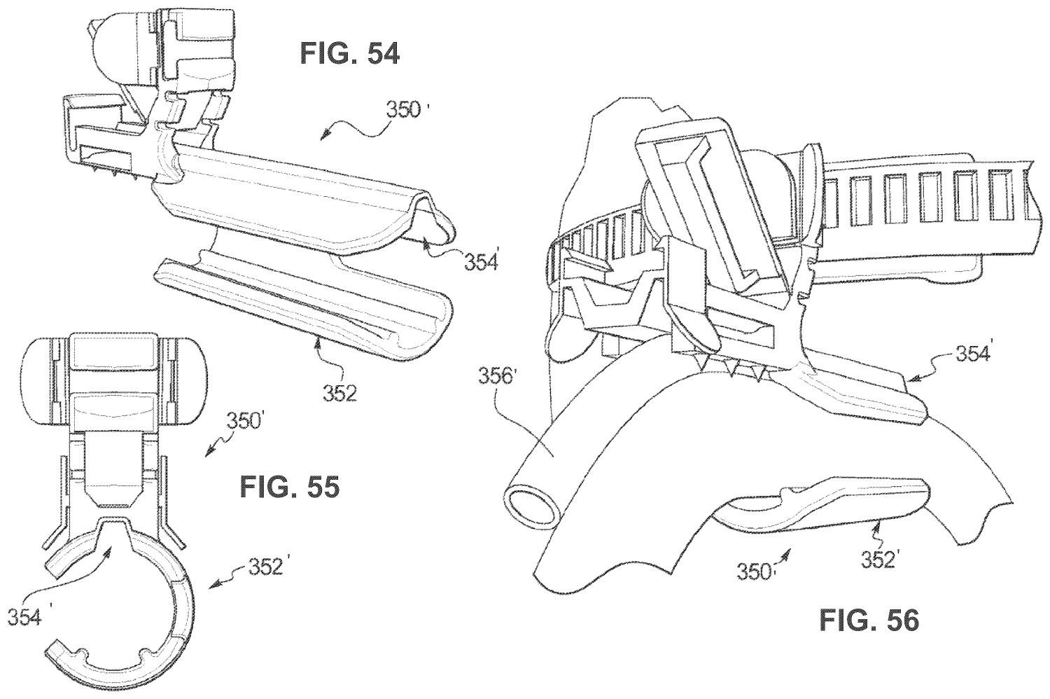

FIGS. 54-56 show still another example of a tube holder 350′ that is substantially the same as many of the prior described example. In this example, the tube holder 350′ has a bite block 352′ that has a raised recess or channel 354′ formed in the upper surface of and extending the length of the bite block 352′. The channel 354′ creates a larger sized passage within the bite block 352′ for an accessory line without significantly affecting the size of ET tube that the bite block 352′ can handle. As shown in FIG. 56, a second accessory line 356′, such as a subglottic suction line, can exit the ET tube and pass within the channel 354′ along with the ET tube.

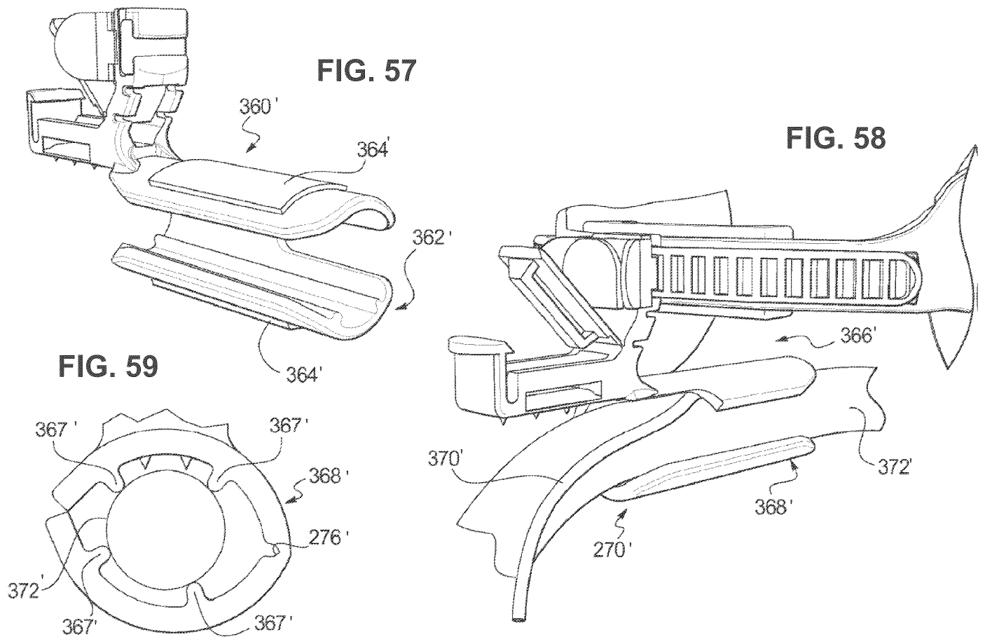

FIG. 57 shows another example of a tube holder 360′ that is essentially the same as the tube holder 40′ described earlier and has a substantially similar bite block 362′. However, in this example, the bite block 362′ has a softer pad 364′ provided on the top and bottom of the bite block 362′. The pads can be adhered to the exterior of the bite block or can be in-molded or dual molded with the bite block but from a softer material. The soft pads 364′ can be added for patient comfort and perhaps safety. The forces applied to the bite block 362′ during use would be at least partly dispersed across the surface of the block via the softer pads 354′. This can minimize stress concentration on the barrel of the bite block and help reduce the likelihood of damage to the patient's dentin while also increasing comfort for those that are awake and actively biting. In another example, the pads 364′ could be combined with the taller bite block 352′ on top of the channel 354′ from the immediately prior example to increase comfort for the patient. The soft material pads 364′ in such an example could be over-molded to cover the raised area of the channel 354′ for the subglottic suction line 356′ or other accessory line to create a soft landing for the patient's upper incisors.

In still another example as shown in FIGS. 58 and 59, a tube holder 366′ that is substantially the same as the earlier described bite block 270′ of FIGS. 43 and 44, with shorter upper ribs 367′ can be used, but have an increased diameter bite block 368′. The tube holder 366′ can be used in conjunction with a larger sized accessory line 370′, such as a subglottic suction tube. The ribs 367′ and line 370′ can be configured to allow for a predetermined amount of compression or crush on the line. The bite block 368′ can also be configured to allow for a predetermined amount of compression or crush on the ET tube 372′. As shown in FIG. 59, and as discussed above, the bite block 368′ can be configured to allow for some compression before the free ends 374′ along the insertion slot 376′ come in contact with one another to resist further compression. The live hinge 378′, similar to the earlier described hinges can help with flexibility in the bite block 368′ to insert the ET tube 372′ and can also help with allowing some limited compression without much resistance.

Any number of the bite block and tube holder features described above can be used in combination, even though such combination is not specifically mentioned herein. Also, one or more features can also be added to any of the bite block examples disclosed herein to aid in inserting an ET tube into the insertion slot and to help prevent discomfort and irritation to the patient during use. As shown in FIG. 30 for example, the forward ends (or the rear ends, if desired) of the free edges 130′, 132′ on the bite block 46′ can include angled or tapered entry segments 310′, 312′ at the leading edge of the insertion slot 128′. The tapered entry segments 310′, 312′ can make the insertion slot 128′ wider, but only at the forward end of the bite block 46′. This can make it easier for medical personnel to spread apart the free edges 130′, 132′ in order to aid in starting to insert an ET tube 44′ into the bite block 46′. Such a feature can be included on any of the disclosed bite blocks and is shown on many of them herein.

In another example, the end face of the bite block need not lie in a plane. Instead, the end face can be curved to form rounded curved contours, especially on the working end of the bite block that will lie within a patient's mouth. For example, FIGS. 25 and 30 show that the bite block 46′ has curved contoured regions 314′ in the axial direction at the working end 124′ of the bite block 46′. Likewise, the exposed end edges of the bite block can also be smooth and curved or rounded. For example, an end edge 316′ of the bite block 200′ shown in FIGS. 36 and 37 are smooth and rounded instead of having sharp corners, particularly at the working end of the bite block. Such features can make the device 30′ more comfortable for a patient during use.

The disclosed ET tube holding device 30′ can be applied or installed on the patient with the ET tube 44′ already positioned in the patient's mouth and trachea. If a temporary bite block device is already prepositioned about the tube, that bite block can be removed and the bite block as described herein can be attached to the ET tube at the same time that the device 30′ is installed. The disclosed bite blocks can be constructed from materials and material thicknesses and with features that render the bite block sufficiently rigid to inhibit the inserted ET tube from being crushed or closed off by a patient's bite during use and yet sufficiently flexible to allow relatively easy insertion and removal of the ET tube as needed.

Although certain ET tube holding devices, features, components, and methods have been described herein in accordance with the teachings of the present disclosure, the scope of coverage of this patent is not limited thereto. On the contrary, this patent covers all embodiments of the teachings of the disclosure that fairly fall within the scope of permissible equivalents.

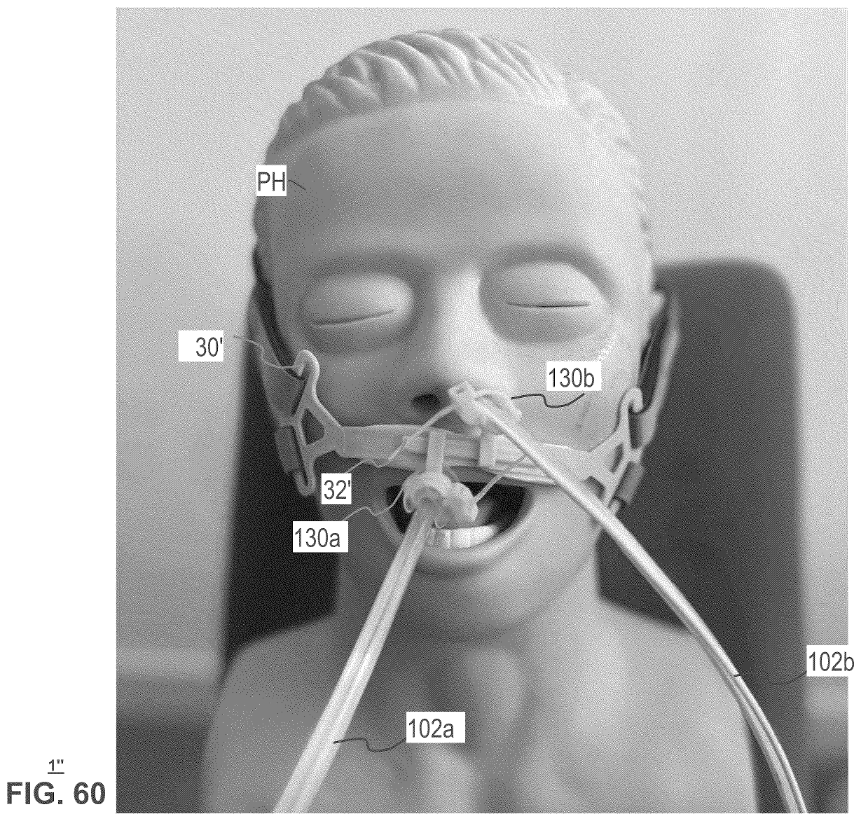

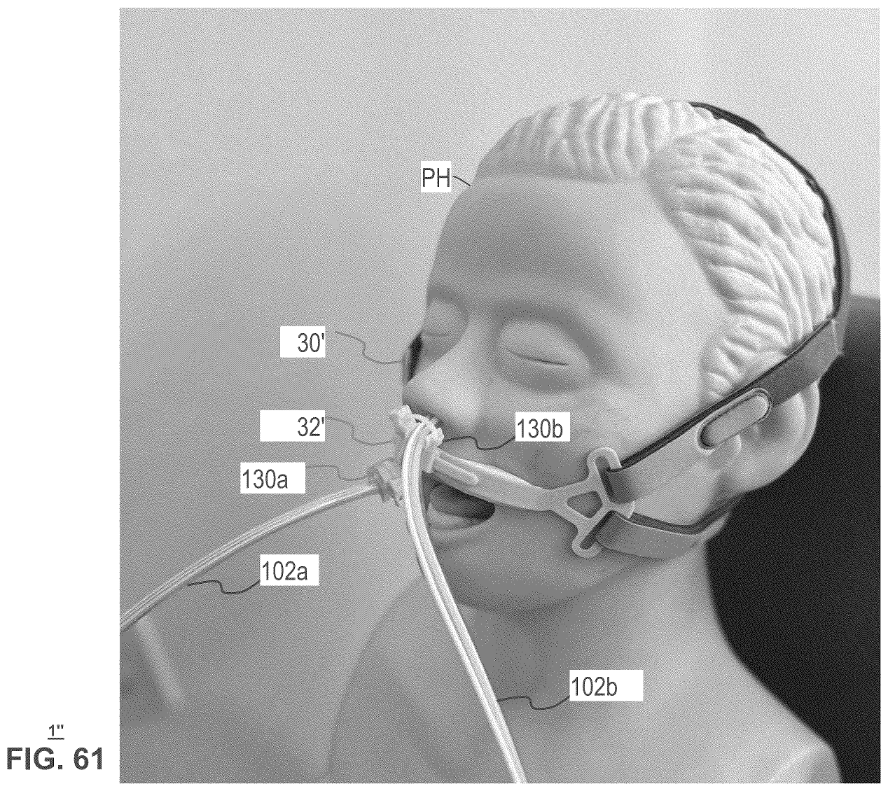

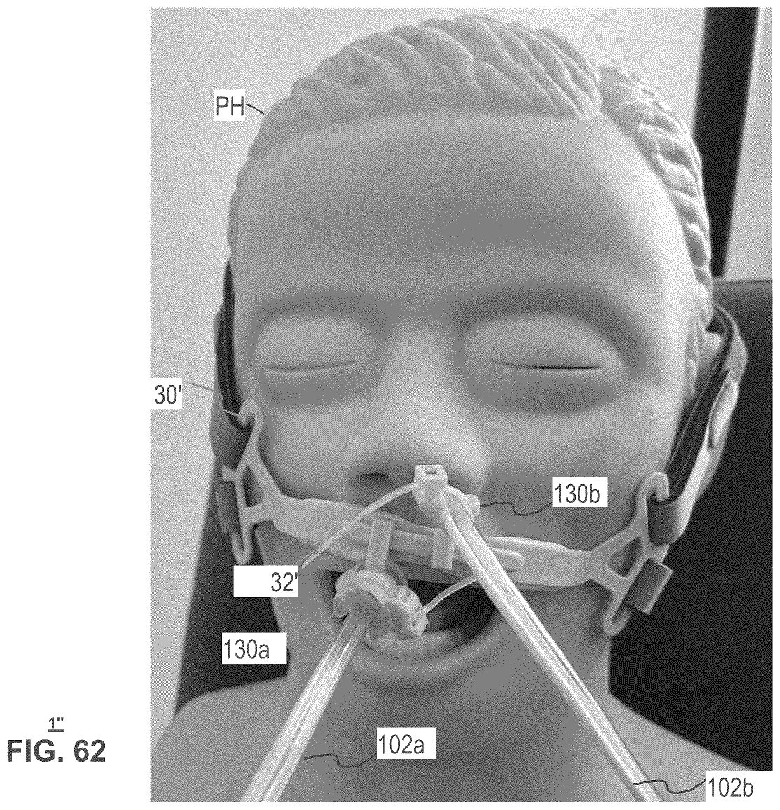

FIGS. 60-62 are different perspective views of a system 1″ that may include patient PH wearing any suitable device (e.g., device 30′ of FIGS. 21-24) with any suitable track (e.g., track 32′ of FIGS. 21-24) and one or more suitable slider lock subassemblies, such as slider lock subassemblies 130a and 130b, one or each of which may be the same or substantially the same as slider lock subassembly 130 of FIGS. 8-11 and may or may not include any suitable tube liner (e.g., tube liner 170). As shown, a portion of a first tube 102a may be held within or by or otherwise coupled to slider lock subassembly 130a, while a portion of a second tube 102b may be held within or by or otherwise coupled to slider lock subassembly 130b of system 1″. Slider lock subassembly 130a of system 1″ may include any suitable slider clamp (e.g., slider clamp 150) that may movably couple slider lock subassembly 130a to track 32′ of device 30′ (e.g., such that slider lock subassembly 130a may be free to slide at least slightly in the direction of one or both of to the left and right with respect to track 32′ when the slider clamp of slider lock subassembly 130a is mated with track 32′ (e.g., the slider clamp 150 of slider lock subassembly 130a may be shaped and sized to be movably coupled to track 32′ in a similar manner to shuttle 60′ and track 32′ and/or similarly to clamp 150 and track body 126)). Additionally, as shown, slider lock subassembly 130b may include any suitable slider clamp (e.g., slider clamp 150) that may movably couple slider lock subassembly 130b to track 32′ of device 30′ (e.g., such that slider lock subassembly 130b may be free to slide at least slightly in the direction of one or both of to the left and right with respect to track 32′ when the slider clamp of slider lock subassembly 130b is mated with track 32′ (e.g., the slider clamp 150 of slider lock subassembly 130b may be shaped and sized to be movably coupled to track 32′ in a similar manner to shuttle 60′ and track 32′ and/or similarly to clamp 150 and track body 126)). Although not shown in FIGS. 60-62, device 30′ of system 1″ may also include any suitable additional tube holder (e.g., tube holder 40′ of FIGS. 25-31 (e.g., with a shuttle 60′)) that may be slidably mounted on track 32′ (e.g., similar to a slider clamp of any one or more slider lock subassemblies 130 of system 1″) in any suitable relationship to slider lock subassembly 130a and/or slider lock subassembly 130b of system 1″ (e.g., between slider lock subassembly 130a and slider lock subassembly 130b along track 32′, to the left of any slider lock subassembly(ies) 130 along track 32′, or to the right of any slider lock subassembly(ies) 130 along track 32′).

In some embodiments, slider lock subassembly 130a and slider lock subassembly 130b may be identical or substantially the same but each may be oriented differently with respect to track 32′ of device 30′ when the slider clamp of the slider lock subassembly is mated with track 32′ of device 30′ of system 1″. For example, as shown, slider lock subassembly 130b may be oriented similar to the orientation of FIG. 10 when its slider clamp (e.g., slider clamp 150) is mated with track 32′ of system 1″, such that a portion of tube 102b may be passed through slider lock subassembly 130b and into a nostril of patient PH (e.g., when the slider clamp of slider lock subassembly 130b is coupled to track 32′ at a position below the nostril), while slider lock subassembly 130a may be oriented 180° to the orientation of FIG. 10 when its slider clamp (e.g., slider clamp 150) is mated with track 32′ of system 1″, such that a portion of tube 102a may be passed through slider lock subassembly 130a and into the mouth of patient PH (e.g., when the slider clamp of slider lock subassembly 130a is coupled to track 32′ at a position above the mouth). Therefore, the tube clamp (e.g., tube clamp 140) of slider lock subassembly 130a can slide (e.g., with the slider clamp (e.g., slider clamp 150) of slider lock subassembly 130a) at least partially below track 32′ (e.g., from the perspective of patient PH) to capture an oral tube (e.g., tube 102a) (e.g., hinge 148h and/or the axis of rotation of slider clamp 150 with respect to tube clamp 140 of slider lock subassembly 130a may be positioned at least partially below track 32′) and/or the tube clamp (e.g., tube clamp 140) of slider lock subassembly 130b can slide (e.g., with the slider clamp (e.g., slider clamp 150) of slider lock subassembly 130b) at least partially above track 32′ (e.g., from the perspective of patient PH) to capture a nasal tube (e.g., tube 102b) (e.g., hinge 148h and/or the axis of rotation of slider clamp 150 with respect to tube clamp 140 of slider lock subassembly 130b may be positioned at least partially above track 32′), while a shuttle of a tube holder (e.g., shuttle 60′ of tube holder 40′ of FIGS. 25-31) may slide along track 32′ to capture a tube (e.g., tube 44′ (e.g., an oral tube) or a nasal tube or the like). Although shown to be supporting tube 102b entering the left nostril of patient PH, slider lock subassembly 130b may be positioned along track 32′ to support tube 102b such that it may enter the right nostril of patient PH, no matter whether slider lock subassembly 130a remains to the right of slider lock subassembly 130b (e.g., as shown in FIGS. 60-62 (e.g., from the perspective of patient PH)) or whether slider lock subassembly 130b is positioned to the right of slider lock subassembly 130a (e.g., from the perspective of patient PH). Any suitable feature(s) may be provided on track 32′ and/or on either one or both of slider lock subassemblies 130a and 130b to prevent a slider lock subassembly from sliding off an end of track 32′ (e.g., all the way to the left and beyond left track end 38′ for disengaging with track 32′ and/or all the way to the right and beyond right track end 38′ for disengaging with track 32′ (e.g., from the perspective of patient PH)). Additionally or alternatively, any suitable feature(s) may be provided on track 32′ and/or on either one or both of slider lock subassemblies 130a and 130b (e.g., by slider clamp 150 and/or by track 32′) and/or on shuttle 60′ to prevent a slider lock subassembly 130 or tube holder 40′ from easily popping off of and away from track 32′ (e.g., out away from the face of the patient) while still enabling an operator (e.g., doctor) to decouple a slider lock subassembly 130 and/or tube holder 40′ from track 32′ and recouple the slider lock assembly 130 and/or tube holder 40′ to track 32′ without sliding the slider lock assembly 130 and/or tube holder 40′ off of an end of track 32′ (e.g., to move slider lock subassembly 130a from a first position along track 32′ between right track end 38′ and slider lock subassembly 130b (e.g., as shown in FIGS. 60-62) to a second position along track 32 between left track end 38′ and slider lock subassembly 130b (e.g., without sliding slider lock subassembly 130a off of right track end 38′ of track 32′)). Therefore, a slider lock subassembly 130 can be positioned on track 32′ before or after any tube holder 40′ may be positioned on track 32′. Slider lock assembly 130 of system 1″ may capture a tube orally or nasally simply by a mere reversal of the orientation of assembly 130 with respect to track 32′ of device 30′ of system 1″. It is to be understood that while FIGS. 60-62 may illustrate subassemblies 130a and 130b oriented differently on track 32′ for enabling tube 102a to enter the mouth while enabling tube 102b to enter a nostril, both subassemblies 130a and 130b may be oriented similarly on track 32′ for enabling tubes 102a and 102b to both enter the mouth or to both enter the same or different nostrils.

As just one example use case, a problem that may be encountered when securing tubes in patients who are intubated (e.g., on a ventilator) is that they may have an endotracheal tube coming out of their mouth that is secured by a device 30′ (e.g., an Anchorfast device by Hollister Incorporated) that may be positioned to sit on a patient's upper lip and wrap around the head, all of which may be taking up a lot of space. Most patients who are intubated for more than 24 hours will likely need a nasogastric tube for feeding, which can be inserted orally or nasally. Securement of such a device has been proven difficult with options such as the tying of the nasogastric tube to the ET tube holder (e.g., tube holder 40′ of device 30′) if its tube (e.g., tube 44′) was placed orogastrically versus an adhesive or using a bridle (e.g., an invasive device) to secure the tubes nasogastrically. The issue that hospitals may face is a high incidence of pressure ulcers as a result of such methods via different mechanisms (e.g., strong acrylic tape that has a stronger bond with the epidermis than the epidermis with the dermis, resulting in skin tears and possible ulcers, or subtle back and forth movement of the tube due to ineffective securement leading to friction and an eventual pressure injury, and/or the like). Therefore, by providing a system 1″ that allows for a tube holder 40′ and one or more slider lock subassembly (ies) 130 that may be repositioned with respect to each other along track 32′ and/or reoriented with respect to track 32′ (e.g., slider lock subassembly 130b as oriented differently with respect to track 32′ as compared to slider lock subassembly 130a and/or a tube holder 40′), a system may be utilized that can easily be adjusted for comfortably accommodating two or more tubes of any suitable type(s) with any suitable relative positions along a track and/or relative orientations with respect to the track and/or the patient.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. The term “and/or” as used herein may refer to and encompass any and all possible combinations of one or more of the associated listed items. As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” may each refer to only A, only B, or only C; any combination of A, B, and C; and/or at least one of each of A, B, and C. The terms “includes,” “including,” “comprises,” and/or “comprising,” when used in this specification, specify the presence of stated features, integers, steps, operations, processes, elements, and/or components, but do not preclude the presence or addition of one or more other features, integers, steps, operations, processes, elements, components, and/or groups thereof. When used in the claims, the term “or” is used as an inclusive or and not as an exclusive or. For example, the phrase “at least one of x, y, or z” means any one of x, y, and z, as well as any combination thereof.

As used herein, the term “or” can be construed in either an inclusive or exclusive sense. Moreover, plural instances can be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and can fall within a scope of various implementations of the present disclosure. In general, structures and functionality presented as separate resources in the example configurations can be implemented as a combined structure or resource. Similarly, structures and functionality presented as a single resource can be implemented as separate resources. These and other variations, modifications, additions, and improvements fall within a scope of implementations of the present disclosure as represented by the appended claims. The specification and drawings are, accordingly, to be regarded in an illustrative rather than a restrictive sense.

The term “if” may, optionally, be construed to mean “when” or “upon” or “in response to determining” or “in response to detecting,” depending on the context. Similarly, the phrase “if it is determined” or “if [a stated condition or event] is detected” may, optionally, be construed to mean “upon determining” or “in response to determining” or “upon detecting [the stated condition or event]” or “in response to detecting [the stated condition or event],” depending on the context.

The predicate words “configured to,” “operable to,” and “operative to” do not imply any particular tangible or intangible modification of a subject, but, rather, are intended to be used interchangeably.

As used herein, the term “based on” may be used to describe one or more factors that may affect a determination. However, this term does not exclude the possibility that additional factors may affect the determination. For example, a determination may be solely based on specified factors or based on the specified factors as well as other, unspecified factors. The phrase “determine A based on B” specifies that B is a factor that is used to determine A or that affects the determination of A. However, this phrase does not exclude that the determination of A may also be based on some other factor, such as C. This phrase is also intended to cover an embodiment in which A may be determined based solely on B. As used herein, the phrase “based on” may be synonymous with the phrase “based at least in part on.”

As used herein, the phrase “in response to” may be used to describe one or more factors that trigger an effect. This phrase does not exclude the possibility that additional factors may affect or otherwise trigger the effect. For example, an effect may be solely in response to those factors, or may be in response to the specified factors as well as other, unspecified factors. The phrase “perform A in response to B” specifies that B is a factor that triggers the performance of A. However, this phrase does not foreclose that performing A may also be in response to some other factor, such as C. This phrase is also intended to cover an embodiment in which A is performed solely in response to B.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter/neutral gender (e.g., her and its and they) and vice versa. Headings and subheadings, if any, are used for convenience only and do not limit the subject disclosure.

While there have been described tube holding assemblies and methods for using and making the same, it is to be understood that many changes may be made therein without departing from the spirit and scope of the subject matter described herein in any way. Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Many alterations and modifications of the preferred embodiments will no doubt become apparent to a person of ordinary skill in the art after having read the foregoing description, it is to be understood that the particular embodiments shown and described by way of illustration are in no way intended to be considered limiting. It is also to be understood that various directional and orientational terms, such as “left” and “right,” “up” and “down,” “front” and “back” and “rear,” “top” and “bottom” and “side,” “above” and “below,” “length” and “width” and “thickness” and “diameter” and “cross-section” and “longitudinal,” “X−” and “Y−” and “Z−,” and/or the like, may be used herein only for convenience, and that no fixed or absolute directional or orientational limitations are intended by the use of these terms. For example, components may have any desired orientation. If reoriented, different directional or orientational terms may need to be used in their description, but that will not alter their fundamental nature as within the scope and spirit of the disclosure.

Therefore, those skilled in the art will appreciate that the concepts can be practiced by other than the described embodiments, which are presented for purposes of illustration rather than of limitation.

1-80. (canceled)

81. A method of using an assembly for holding a tube and another tube to a patient's head once a distal end of the tube has been functionally positioned within the patient via a first nostril of a nose of the patient, the method comprising:
positioning a mount subassembly of the assembly such that a track of the positioned mount subassembly extends above and along an upper lip of the patient;
sliding a slider clamp body of a lock subassembly of the assembly along the track of the positioned mount subassembly until a tube clamp body of the lock subassembly is aligned with a portion of the tube;
rotating the tube clamp body with respect to the slid slider clamp body until a passageway of the tube clamp body is aligned with the portion of the tube;
placing the portion of the tube within the passageway of the rotated tube clamp body; and
sliding another slider clamp body of another lock subassembly of the assembly along the track of the positioned mount subassembly until another tube clamp body of the other lock subassembly is aligned with at least one of:
a portion of the other tube;
a second nostril of the nose of the patient; or
a mouth of the patient.

82. The method of claim 81, wherein:
the tube is a tracheal tube; and
the other tube is a gastric tube.

83. The method of claim 81, wherein, once a distal end of the other tube has been functionally positioned within the patient via the mouth of the patient, the sliding the other slider clamp body comprises sliding the other slider clamp body of the other lock subassembly of the assembly along the track of the positioned mount subassembly until the other tube clamp body of the other lock subassembly is aligned with a portion of the other tube.

84. An assembly for holding a tube and another tube to a patient's head, the assembly comprising:
a mount subassembly defining a track;
a lock subassembly comprising:
a slider clamp body operative to be coupled to and slidable along the track;
a tube clamp body operative to receive and hold a portion of the tube in place with respect to the tube clamp body; and
a hinge operative to allow rotation of the slider clamp body with respect to the tube clamp body about an axis of rotation;
another lock subassembly comprising:
another slider clamp body operative to be coupled to and slidable along the track;
another tube clamp body operative to receive and hold a portion of the other tube in place with respect to the other tube clamp body; and
another hinge operative to allow rotation of the other slider clamp body with respect to the other tube clamp body about another axis of rotation; and
a fastener subassembly operative to secure the mount subassembly to the patient's head.

85. The assembly of claim 84, wherein, when the slider clamp body is coupled to the track and the other slider clamp body is coupled to the track, the axis of rotation is above the track and the other axis of rotation is below the track.

86. The assembly of claim 84, wherein, when the slider clamp body is coupled to the track, the axis of rotation is above the track.

87. The assembly of claim 84, wherein, when the other slider clamp body is coupled to the track, the other axis of rotation is below the track.

88. The assembly of claim 84, wherein the hinge is operative to allow at least 180 degree rotation of the slider clamp body with respect to the tube clamp body about the axis of rotation.

89. The assembly of claim 88, wherein the other hinge is operative to allow at least 180 degree rotation of the other slider clamp body with respect to the other tube clamp body about the other axis of rotation.

90. The assembly of claim 84, wherein:
the tube is a tracheal tube; and
the other tube is a gastric tube.

91. An assembly for holding a tube and another tube to a patient's head, the assembly comprising:
a mount subassembly comprising:
a base; and
a track extending from and along a front face of the base;
a lock subassembly comprising:
a slider clamp body operative to be coupled to and slidable along the track;
a tube clamp body operative to be coupled to the tube; and
a hinge operative to allow movement of the slider clamp body with respect to the tube clamp body;
another lock subassembly comprising:
another slider clamp body operative to be coupled to and slidable along the track;
another tube clamp body operative to be coupled to the other tube; and
another hinge operative to allow movement of the other slider clamp body with respect to the other tube clamp body; and
a strap subassembly operative to secure the mount subassembly to the patient's head.

92. The assembly of claim 91, wherein:
the tube clamp body defines a tube clamp passageway along and within which the tube clamp body is operative to hold a portion of the tube in place with respect to the tube clamp body; and
when the slider clamp body is coupled to the track, the assembly is operative to allow the tube clamp passageway to extend simultaneously transverse to the track and in front of the front face of the base.

93. The assembly of claim 91, wherein:
the tube clamp body defines a tube clamp passageway along a length of an interior of the tube clamp body extending between a tube clamp body top and a tube clamp body bottom;
the tube clamp body is operative to hold a portion of the tube in place with respect to the tube clamp body within the tube clamp passageway and along the length of the interior of the tube clamp body;
the tube clamp passageway is accessible by the portion of the tube via a tube clamp body opening extending through a front face of the tube clamp body and between the tube clamp body top and the tube clamp body bottom;
the tube clamp body opening is operative to introduce the portion of the tube into the tube clamp passageway; and
when the slider clamp body is coupled to the track, the hinge is operative to allow movement of the slider clamp body with respect to the tube clamp body such that the tube clamp passageway is positioned between the tube clamp body opening and the track.

94. The assembly of claim 93, wherein the hinge is operative to allow movement of the slider clamp body with respect to the tube clamp body such that the tube clamp body opening faces away from the slider clamp body when the slider clamp body is coupled to the track.

95. The assembly of claim 91, wherein the hinge is operative to allow movement of the slider clamp body with respect to the tube clamp body such that the tube clamp body opening faces away from the slider clamp body when the slider clamp body is coupled to the track.

96. The assembly of claim 91, wherein the hinge is operative to allow rotation of the slider clamp body with respect to the tube clamp body about an axis of rotation.

97. The assembly of claim 96, wherein the other hinge is operative to allow rotation of the other slider clamp body with respect to the other tube clamp body about another axis of rotation.

98. The assembly of claim 97, wherein, when the slider clamp body is coupled to the track and the other slider clamp body is coupled to the track, the axis of rotation is above the track and the other axis of rotation is below the track.

99. The assembly of claim 96, wherein the hinge is operative to allow at least 180 degree rotation of the slider clamp body with respect to the tube clamp body about the axis of rotation.

100. The assembly of claim 91, wherein:
the tube is a tracheal tube; and
the other tube is a gastric tube.
